(19) United States
(12) Patent Application Publication
(10) Pub. No.: US 2009/0246756 A1
(43) Pub. Date: Oct. 1, 2009

(54) Thermostable polypeptide having polynucleotide kinase activity and/or phosphatase activity

(75) Inventors: Thorarinn Blondal, Gardabaer (IS); Arnthor Aevarsson, Hveragerdi (IS); Gudmundur O. Hreggvidsson, Reykjavik (IS); Jakob Kristjansson, Gardabaer (IS)

Correspondence Address: BIRCH STEWART KOLASCH & BIRCH, PO BOX 747, FALLS CHURCH, VA, 22040-0747, US

(73) Assignee: Prokaria Ehe, Reykjavik (IS)

(21) Appl. No.: 11/579,733

(22) PCT Filed: May 6, 2005
(86) PCT No.: PCT/IS05/00011

§ 371 Date: Feb. 1, 2007

(30) Foreign Application Priority Data
May 6, 2004 (IS) 7249

(51) Int. Cl. C12Q 1/68; C12N 9/12; C12N 9/16; C12P 19/34

(52) U.S. Cl. 435/6; 435/194; 435/196; 435/91.53

(57) ABSTRACT

Isolated polypeptides having 5′-kinase and/or 3′-phosphatase activity and temperature optimum of at least 60° C. are described. The invention also relates to isolated nucleic acids encoding the polypeptides, nucleic acid constructs and host cells comprising the nucleic acid sequences as well as methods using the polypeptides and kits for practicing the methods.

### BACKGROUND OF THE INVENTION

Polynucleotide kinase (PNK) from bacteriophage T4 is a widely used tool in molecular biology today and is used for example for labelling of nucleic acid with radioactive chemical groups to enable subsequent detection. Bacteriophage T4 PNK has two activities on nucleic acids; a 5′ kinase activity and 3′ phosphatase activity. The enzyme thus catalyses the removal of a phosphate group from a 3′ end and the addition of phosphate group to a 5′ end. It is believed that the natural role of the enzyme is to act together with T4 RNA ligase 1 to counteract suicide reaction of the host by repairing tRNA molecules that have been cut at the anticodon loop by host cell anticodon nuclease activated by the viral infection. The anticodon nuclease cleaves tRNALys 5′ to its wobble position yielding 2′-3′ cyclic phosphate and a 5′ hydroxyl group. While the T4 RNA ligase 1 is essential for ligation of the degraded tRNA molecules, the T4 PNK has the important role of making the tRNA fragments appropriate substrates for the ligation step. The T4 PNK thus removes the 2′-3′ cyclic phosphate from the 5′ tRNA fragment and adds a phosphate group to the 5′ hydroxyl group of the 3′ tRNA fragment using ATP as the phosphate donor. The RNA ligase 1 and the polynucleotide kinase are thus part of the same system, acting in concert to repair a dysfunctional translational machinery.

T4 RNA ligase is also widely used in various applications and T4 RNA ligase and T4 PNK are sometimes used in the same procedure. However, although T4 PNK can be utilized for various useful applications, it is only functional to about 40° C.

Recently, a thermostable RNA ligase (homologous to the T4 RNA ligase 1) from the thermophilic bacteriophage RM378 that infects the thermophilic eubacterium Rhodothermus marinus was described (Blondal, T. et al. (2003) Nucleic Acids Res 31, 7247-7254). However, a thermostable PNK has not been described to date. The T4 PNK is the first defined member of a large family of 5′ kinases/3′ phospho-hydrolases that have been discovered. The family includes polynucleotide kinases from a wide variety of organisms. The biological role of the eukaryotic 5′-kinase/3′-phosphohydrolases is to mend broken nucleic acids strands, making them appropriate substrates for repair by appropriate nucleic acid ligase, thereby playing an important role, for example in DNA repair of nicks and gaps (Jilani, A. et al. (1999) J Cell Biochem 73, 188-203, Meijer, M. et al. (2002) J Biol Chem 277, 4050-4055; Karimi-Busheri, F. et al. (1999) J Biol Chem 274, 24187-24194). Sequence and mutational analysis have shown that T4 PNK is a homo-tetramer although any kinetic cooperativity has not been demonstrated (Lillehaug, J. R., and Kleppe, K. (1975) Biochemistry 14, 1221-1225; Wang, L. K. et al. (2002) Embo J 21, 3873-3880; Wang, L. K., and Shuman, S. (2001) J Biol Chem 276, 26868-26874; Galburt, E. A. et al. (2002) Structure (Camb) 10, 1249-1260). The 5′ kinase and 3′ phosphohydrolase activities have been shown to reside in distinct domains, with N-terminal 5′ kinase domain and a C-terminal 3′ phosphohydrolase domain. The 5′ kinase domains of polynucleotide kinases contain a nucleotide binding motif (commonly referred to as “Walker A box” or “P-loop”) with the signature GXXXXGK(S/T) (X denotes any amino acid) which is a common motif in phosphotransferases as well as other nucleotide binding domains (Wang, L. K. et al. (2002) Embo J 21, 3873-3880; Midgley, C. A., and Murray, N. E. (1985) Embo J 4, 2695-2703). Mutational data from T4 PNK has shown that in addition to residues in the P-loop motif (K15 and S16), residues D35, R38, D85 and R126 are all essential for the 5′ kinase activity (Wang, L. K., and Shuman, S. (2002) Nucleic Acids Res 30, 1073-1080). In addition, studies have suggested a role of residues D85 and N87 in the quaternary structure integrity. When these residues are changed by site directed mutagenesis a mixture of dimmers and tetramers is obtained (Wang, L. K. et al. (2002) Embo J 21, 3873-3880; Wang, L. K., and Shuman, S. (2001) J Biol Chem 276, 26868-26874; Wang, L. K., and Shuman, S. (2002) Nucleic Acids Res 30, 1073-1080).

The sequence analysis and mutational data on the 3′ phosphohydrolase domain in T4 PNK have shown that the metal-dependent phosphatase family motif DXDXT is found in the PNK family, and is essential for the phosphohydrolase activity of the domain (Wang, L. K. et al. (2002) Embo J 21, 3873-3880; Wang, L. K., and Shuman, S. (2001) J Biol Chem 276, 26868-26874; Wang, L. K., and Shuman, S. (2002) Nucleic Acids Res 30, 1073-1080). Sequence analysis of PNK show that the 3′ phosphatase domain of T4 PNK is distantly related to other phosphatase families like the histidinol phosphatase family involved in metabolic pathways and Acid phosphatase (HAD) superfamily. The crystal structure of the T4 PNK was solved by Galburt et al. and confirmed that there were two functionally distinct structural domains. The N-terminal 5′ kinase domain is structurally similar to adenylate kinase and the 3′ phosphatase domain shows structural similarity to members of the superfamily of HAD hydrolases (Galburt, E. A. et al. (2002) Structure (Camb) 10, 1249-1260).

Enzymes from thermophiles are often more suitable for industrial processes than their mesophilic counterparts. Thermostable enzymes are used in various commercial settings such as proteases and lipases used in washing powder, hydrolytic enzymes used in bleaching and glycosyl hydrolases used in the food industry. The use of thermostable enzymes, foremost thermostable DNA polymerases, has also revolutionized the field of recombinant DNA technology and is of great importance in the research industry today. Identification of new thermophilic enzymes in particular thermophilic nucleic acid-modifying enzymes will facilitate continued research as well as assist in improving commercial enzyme-based products. Enzymes of this kind may proof to be valuable tools in various applications in recombinant DNA technology and other molecular biology procedures.

### BRIEF DESCRIPTION OF THE DRAWINGS

The foregoing and other objects, features and advantages of the invention will be apparent from the following more particular description of preferred embodiments of the invention, as illustrated in the accompanying drawings, in which:

FIG. 1 is the nucleic acid sequence of the open reading frame (ORF) encoding a polynucleotide kinase from bacteriophage RM378 (SEQ ID NO: 1).

FIG. 2 is the amino acid sequence of the polynucleotide kinase from bacteriophage RM378 (SEQ ID NO: 2).

FIG. 3 shows amino acid sequence alignment of the phosphohydrolase domain (HD domain) and the kinase domain. (A) The phosphohydrolase HD domain of RM378 PNK, C. acetobutylicum putative polyA polymerase and D. hafniense tRNA nucleotidyltransferase/poly(A) polymerase. Note that sequences have been truncated and the alignment is shown only over the HD domain. The HD motif is boxed. Sequence identity of the HD domain in RM378 compared to C. acetobutylicum HD polyA polymerase and D. hafniense tRNA nucleotidyltransferase/poly(A) polymerase is 28% and 24%, respectively, over the aligned region. (B) The 5′-kinase domain of RM378 PNK, T4 PNK and Mycobacteriophage Cjw1 PNK. Note that sequences have been truncated and the alignment is shown only over the kinase domain. The P-loop motif is boxed. Sequence identity of RM378 5′-kinase domain when compared to T4 and Cjw1 domains is 15% and 20%, respectively, over the aligned region. The sequences of RM378 phosphohydrolase HD domain in (A) and 5′-kinase domain in (B) are shown with an overlap of a few residues (residues 174 to 178).

FIG. 4 shows purification of RM378 PNK on His-tag column chromatography using imidazole step gradient. Lane 1; Size marker. Lane 2; Crude extract. Lane 3; Flow through. Lane 4; 15% imidazole (500 mM stock solution) step wash. Lanes 5-9: 40% imidazole (500 mM stock solution) elution. Purification estimated at 95% by SDS-PAGE analysis.

FIG. 5 illustrates some characteristics of the PNK 5′ kinase. (A) A pH profile of the enzyme activity shows optimum activity between pH 8 and 9. (B) Relative temperature optimum of the 5′ kinase activity is between 60 and 70° C. (C) The 5′ kinase activity over time 50° C. (filled squares), 60° C. (open squares), 65° C. (open diamonds) and 70° C. (filled diamonds). The enzyme showed relatively good thermostability up to 65° C. but started to loose activity at higher temperatures. (D) PEG 6000 strongly enhanced the 5′ kinase activity at 5-10% concentration, resulting in 4-fold increase in activity.

FIG. 6A shows the effect of ADP on the 5′ kinase activity of 5′ hydroxylated single-stranded DNA (ssDNA) (black bars), dephosphorylation of 5′phosphorylated ssDNA (white bars) and phosphate exchange reaction (grey bars). The 5′kinase reaction was inhibited as the concentration of ADP increased. The dephosphorylation increased as the ADP concentration increased and the exchange reaction was constant and unaffected by the ADP concentration. FIG. 6B shows a Lineweaver-Burk plot of effect of ATP concentration on the 5′ kinase reaction. Kₘ for the ATP was calculated 20 μM. FIG. 6C shows a Lineweaver-Burke plot of the 5′ kinase activity on different concentrations of DNA (filled squares) and RNA (filled diamonds). Vₘₐₓ was calculated to be 160 and 220 μmol*h⁻¹*mg⁻¹ for DNA and RNA respectively. The Kₘ constants were 1.5 and 1.3 μM for DNA and RNA respectively for the given reaction. FIG. 6D shows denaturing polyacrylamide gel electrophoresis (20%) on 5′ kinase labelling reaction of 20 μM d(A20) and r(A20) with 10 μM gamma ³²P-labeled ATP, using 0.2,1 and 5 units of RM378 PNK at 70° C. for 30 min. The labelling was very efficient and completely depleted the ATP when using 5 U per reaction, for both RNA and DNA substrates.

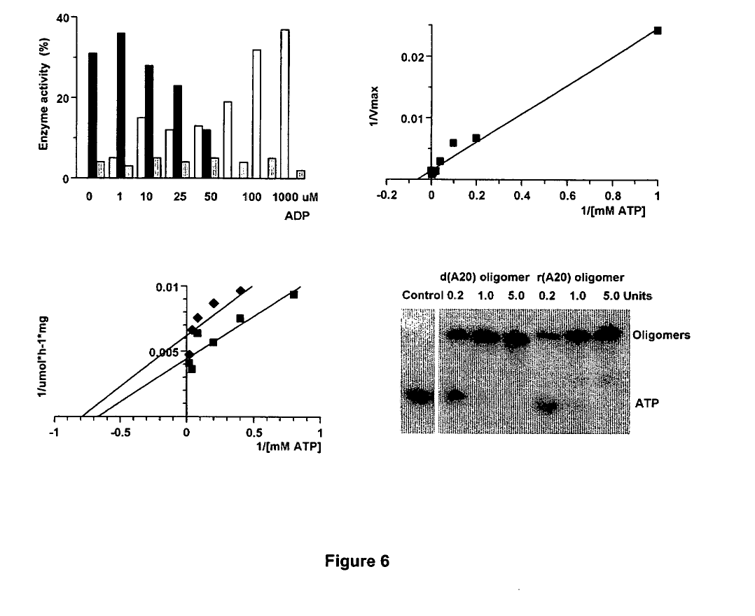

FIG. 7 illustrates characterization of the 3′ phosphohydrolase activity of RM378 PNK. (A) A pH profile, measured in potassium acetate buffer at pH 4-6 (squares) and in MOPS buffer at pH 6-9 (diamonds). Optimum was determined to be close to pH 6. (B) phosphohydrolase activity as function of temperature; optimum was 75° C. measured for one hour, but the enzyme is not stable for extended time at temperatures higher than 65° C. (C) titration of CAMP (squares) and 3′TMP (diamonds), under standard assay conditions with Mn2+ as the cation, clearly shows that the 3′ hydrolase activity is several-fold higher on CAMP than 3′TMP. (D) Comparison of T4 and RM378 PNK 3′phosphohydrolase activity using 0.1 mM CAMP (white bar), 3′TMP (black bar) and d(A15)-3′PO4− oligomer using Mn2+ or Mg2+ as cation for the reaction. The substrate specificity of the two enzymes are clearly different, RM378 showing high activity on CAMP and some activity on 3′TMP but no activity on the oligomer, and little activity in presence of Mg2+. On the other hand, T4 PNK has similar activity on 3′TMP and the oligomer but much less activity on the CAMP.

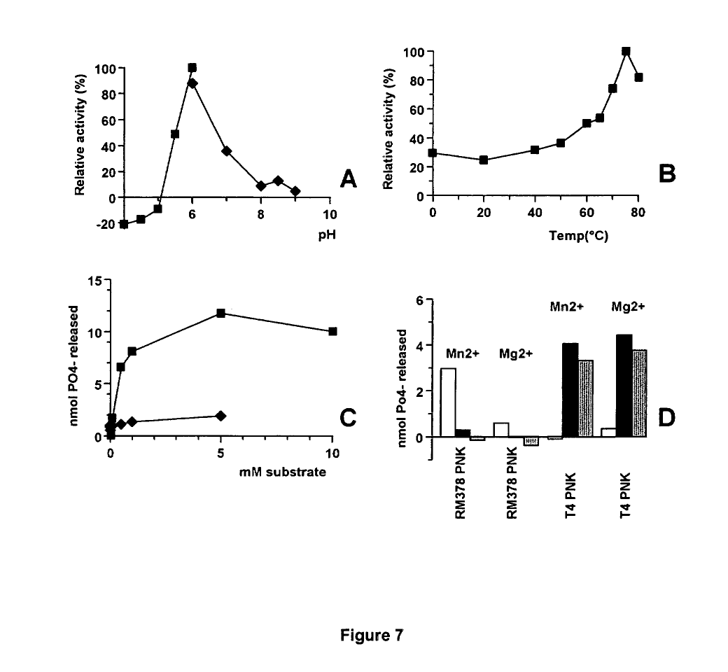

### SUMMARY OF THE INVENTION

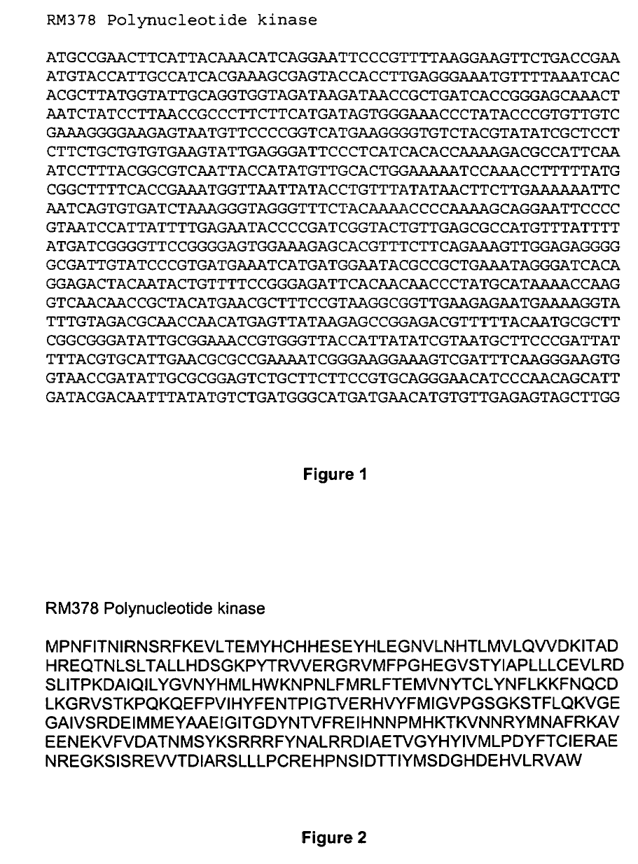

The present invention relates to isolated polypeptides having 5′-kinase and/or 3′-phosphatase activity and preferably having both activities as well as active derivatives or fragments thereof, i.e. derivatives and fragments retaining the 5′-kinase and/or the 3′-phosphatase activity and preferably having both activities. The invention encompasses the polypeptide having the amino acid sequence shown as SEQ ID NO: 2 and polypeptides having 5′-kinase and/or 3′-phosphatase activity with substantially similar amino acid sequences to the sequence as shown in SEQ ID NO: 2 or active derivatives or fragments thereof. The invention further pertains to nucleic acids encoding the polypeptides of the invention. One such nucleic acid is shown in FIG. 1, also shown as SEQ ID NO: 1. The invention also pertains to DNA constructs containing the isolated nucleic add molecules operatively linked to a regulatory sequence; and to host cells comprising the DNA constructs.

This invention pertains in one embodiment to isolated thermostable polypeptides having 5′-kinase and/or 3′-phosphatase activity which are derived from bacteriophages that infect thermophilic bacteria. In certain embodiments, the invention relates to isolated thermostable polypeptides having 5′-kinase and/or 3′-phosphatase activity, which are derived from bacteriophage that infect the bacteria Rhodothermus marinus. Isolated polypeptides provided by the invention can replace T4 polynucleotide kinase in applications that utilize T4 polynucleotide kinase and may also be used in other applications, in particular applications that require elevated temperatures (above about 50° C.).

In one embodiment of the invention, the isolated thermostable polypeptide having 5′-kinase and 3′-phosphatase activity provided by the invention refers to a novel polynucleotide kinase (PNK) from the thermophilic bacteriophage RM378. Compared to T4 PNK, the RM378 PNK has analogous activity but novel phosphohydrolase domain of a different origin and different domain architecture with reversed order of the two principal domains of the polypeptide.

The polypeptides of the invention have been found to be significantly more thermostable than other homologous polypeptides known in the prior art, such as polynucleotide kinase from bacteriophage T4. The enhanced stability of the polypeptides provided by the invention allow their use under temperature conditions which would be prohibitive for some other analogous enzymes, thereby increasing the range of conditions which can be employed and also the type of methods that can be used. Additionally, the polypeptides of the invention have other different functional properties that can be advantageous in certain applications, compared to other homologous polypeptides known from prior art, such as polynucleotide kinase from bacteriophage T4.

The invention further pertains to the use of the polypeptides provided by the invention in various applications including nucleotide labelling, oligonucleotide synthesis and gene synthesis.

The invention pertains to a method of transferring a phosphate group or phosphate analogues from nucleotide triphosphate or nucleotide analogues triphosphates to 5′ ends of nucleic acids or nucleic acid analogues using an isolated thermostable polypeptide having 5′-kinase and/or 3′-phosphatase activity. In certain embodiments, the thermostable polypeptide having 5′-kinase and/or 3′-phosphatase activity can be derived from a thermostable bacteriophage; the nucleic acids can be RNA or DNA; the RNA or DNA can be single-stranded; and the nucleotide analogues may contain modified bases, modified sugars and/or modified phosphate groups.

In yet another embodiment, a method of synthesizing an oligonucleotide polymer by repeating cycles of combining a primer oligonucleotide and a blocked oligonucleotide is described, the method comprising: a) combining the primer oligonucteotide and an oligonucleotide blocked at the 3′ or 5′ end in the presence of a RNA ligase, thereby forming an extended primer with a blocked 3′ or 5′ end; b) removing the blocked phosphate group at the 3′ or 5′ end or adding a phosphate group to the 5′ end of the extended primer using thermostable polypeptides having 5′-kinase and/or 3′-phosphatase activity; and c) repeating a) and b) using the extended primer from b) as the primer for a) wherein an oligonucleotide polymer is formed. In certain embodiments, the formed oligonucleotide polymer comprises a gene or a part of a gene coding for a polypeptide.

Also provided by the invention are kits for practicing the subject methods. In further describing the subject invention, the subject methods will be discussed first in greater detail followed by a description of the kits for practicing the subject methods.

A thermostable polypeptide having 5′-kinase and/or 3′-phosphatase activity of the present invention is suitably selected from the group consisting of: a thermostable polypeptide having 5′-kinase and/or 3′-phosphatase activity obtained from a bacteriophage infecting a thermophilic bacteria; a polypeptide comprising the amino acid sequence of SEQ ID NO: 2; a polypeptide encoded by a nucleic acid comprising the sequence of SEQ ID NO: 1; a polypeptide having at least 30% sequence identity with the amino acid sequence of SEQ ID NO: 2 which retains at least the 5′-kinase activity or 3′-phosphatase activity; or an active fragment or derivative thereof, i.e. retaining either or both the 5′-kinase and 3′-phosphatase activity.

The thermostable polypeptides having 5′-kinase and/or 3′-phosphatase activity described herein have advantageous properties in comparison to prior art PNK-ases including the T4 PNK, such as different substrate specificity and ability to provide activity at relatively high temperatures. In particular embodiments, methods utilizing thermostable polypeptides having 5′-kinase and/or 3′-phosphatase activity are performed at temperatures of at least 50° C., such as at least 60° C. In a preferred embodiment, the methods of the invention are performed at temperatures in the range of about 50° C. to about 95° C. such as the range of about 50° C. to about 75° C.

### DETAILED DESCRIPTION OF THE INVENTION

The present invention relates to isolated thermostable polypeptides having 5′-kinase and/or 3′-phosphatase activity and active derivatives or fragments thereof, i.e. which either of band preferably both the 5′-kinase and 3′-phosphatase activity. The invention encompasses the polypeptide having the amino acid sequence shown as SEQ ID NO: 2 and polypeptides having 5′-kinase and/or 3′-phosphatase activity with substantially similar amino acid sequences to the sequence as shown in SEQ ID NO: 2 or derivatives or fragments thereof which retain at least either of said 5′-kinase and 3′-phosphatase activities. The polypeptide comprising the sequence shown in SEQ ID NO: 2 has limited sequence identity to known sequences deposited in a public database (non redundant protein sequence database of the National Center for Biotechnology Information). Apparently, the polypeptide does thus not have more than 30% sequence identity overall to any polypeptide sequence known in the prior art. The invention encompasses isolated thermostable polypeptides having 5′-kinase and/or 3′-phosphatase activity and having more than 30% sequence identity to SEQ ID NO: 2 and preferably more than 40% or more preferably more than 50% sequence identity to SEQ ID NO: 2 and yet more preferably more than 60% or more than 70% sequence identity to SEQ ID NO: 2. The invention further pertains to nucleic acids encoding the polypeptides of the invention. One such nucleic acid is shown in FIG. 1. The invention also pertains to DNA constructs containing the isolated nucleic acid molecules operatively linked to a regulatory sequence; and to host cells comprising the DNA constructs.

This invention pertains to isolated thermostable polypeptides having 5′-kinase and/or 3′-phosphatase activity, which are derived from bacteriophages that infect thermophilic bacteria. In certain embodiments, the invention relates to isolated thermostable polypeptides having 5′-kinase and/or 3′-phosphatase activity, which are derived from bacteriophage that infect the bacteria Rhodothermus marinus.

Thermophilic polypeptides having 5′-kinase and/or 3′-phosphatase activity or any substantially similar polypeptide encompassed by the present invention are preferably selected from the group consisting of:
a) a thermostable polypeptides having 5′-kinase and/or 3′-phosphatase activity obtained from a bacteriophage infecting a thermophilic bacteria;
b) a polypeptide comprising the amino acid sequence of SEQ ID NO: 2;
c) a polypeptide encoded by a nucleic acid comprising the sequence of SEQ ID NO: 1;
d) a polypeptide having at least 30% sequence identity with the amino acid sequence of SEQ ID NO: 2;
e) a fragment or derivative of (a), (b), (c) or (d).

Isolated polypeptides provided by the invention can replace T4 polynucleotide kinase in applications that utilize T4 polynucleotide kinase. The invention provides in one embodiment a novel polynucleotide kinase (PNK) from the thermophilic bacteriophage RM378. Compared to T4 PNK, the RM378 PNK has analogous activity but novel phosphohydrolase domain of a different origin and different domain architecture with reversed order of the two principal domains of the polypeptide.

The polypeptides of the invention have been found to be significantly more thermostable than other homologous polypeptides known in the prior art, such as polynucleotide kinase from bacteriophage T4.

Polynucleotide kinase (PNK) is defined herein as an enzyme which has one or both of two enzyme activities: a 5′-kinase activity and/or a 3′-phosphatase activity. An enzyme having a 5′-kinase activity is an enzyme that catalyzes the transfer of the gamma-phosphate of a nucleoside 5′-triphosphate to the 5′-hydroxyl terminus of a ribonucleic acid or a deoxyribonucleic acid. The nucleic acid substrates can be a nucleoside 3′-phosphate, an oligonucleotide or a polynucleotide. The reaction produces a nucleoside 5′-diphosphate and a 5′-phosphoryl-terminated nucleotide, oligonucleotide or polynucleotide. The enzyme may also catalyze phosphorylation of modified nucleic acids, such as by containing nucleotides with bases containing chemically protected groups. An enzyme having a 3′-phosphatase activity is an enzyme that catalyzes the hydrolysis of 3′phosphoryl groups on nucleic acids such as nucleoside 3′-monophosphates, including cyclic nucleoside monophosphates, nucleoside 3′,5′-diphosphates or 3′-phosphoryl polynucleotides. The reaction produces a inorganic orthophosphates and a 3′-hydroxyl group (Richardson C. C. (1981), in The enzymes vol XIV, P. D. Boyer, ed. Volume 14 (Academic Press, San Diego), pp. 299-314).

The 5′-kinase activity may be determined by an appropriate assay such as an assay developed for T4 PNK that measures conversion of ATP labelled with radioactive gamma-phosphate group (Richardson, C. C. (1965) Proc Natl Acad Sci U.S.A. 54, 158-165). The standard assay may be modified according to the requirement of a specific PNK. For example, for RM378 PNK the typical reaction conditions are 50 mM MOPS buffer pH 8.5, 1 mM DTT, 10 mM MgCl2, 25 μg/ml BSA, 1 mM spermidine and 5% PEG6000, 100 μM ATP (mixture of normal and γ-32P-ATP) and 0.5 mg/ml partial micrococcal nuclease digested calf thymus DNA or 50-100 μM DNA/RNA oligomers, and 0.0001-0.001 mg/ml PNK enzyme. The reaction mixture is then incubated at 70° C. for 15-30 minutes.

The 3′-phosphatase activity may be determined by an appropriate assay using appropriate substrate, such as developed for T4 PNK (Becker, A. & Hurwitz, (1967), J. Biol. Chem. 242:936-950). For example, the following assay can be used: in a potassium acetate buffer, pH 6.0, with 5 mM MnCl₂, 1 mM DTT, and 10 mM KCl₂, the enzyme is incubated (at 0.05 mg enzyme/ml) with substrate, such as 0.1-5 mM 3′-thymidine mono-phosphate (3′-TMP) or cyclic 2′-3′cyclic adenosine mono-phosphate (cAMP), for 30-60 minutes at suitable temperature, for example at 70-75° C. for RM378 kinase. The reaction is then quenched by adding 90 μL of Biomol Green reagent (Biomol Research Laboratories, Plymouth Meeting, Pa.) to 10 μL reaction. The release of phosphate is then measured at A 620 nm in a spectrophotometer and compared to a phosphate standard curve.

As used herein, “nucleobase” refers to a nitrogen-containing heterocyclic moiety, e.g., a purine, a 7-deazapurine, or a pyrimidine. Typical nucleobases are adenine, guanine, cytosine, uracil, thymine, 7-deazaadenine, 7-deazaguanine, and the like.

“Nucleoside” as used herein refers to a compound consisting of a nucleobase linked to the C-1′ carbon of a ribose sugar.

“Nucleotide” as used herein refers to a phosphate ester of a nucleoside, as a monomer unit or within a nucleic acid. Nucleotides are sometimes denoted as “NTP” or “dNTP” and “ddNTP” to particularly point out the structural features of the ribose sugar.

“Nucleotide 5′-triphosphate” refers to a nucleotide with a triphosphate ester group at the 5′ position. The triphosphate ester group can include sulphur substitutions for the various oxygen atoms, e.g., alpha-thio-nucleotide 5′-triphosphates.

As used herein, the term “nucleic acid” encompasses the terms “oligonucleotide” and “polynucleotide” and means single-stranded or double-stranded polymers of nucleotide monomers, including 2′-deoxyribonucleotides (DNA) and ribonucleotides (RNA). The nucleic acid can be composed entirely of deoxyribonucleotides, entirely of ribonucleotides, or chimeric mixtures thereof, linked by internucleotide phosphodiester bond linkages, and associated counter-ions, e.g., H⁺, NH₄⁺, trialkylammonium, Mg²⁺, Na⁺ and the like. The nucleic acid may also be a peptide nucleic acid (PNA) formed by conjugating bases to an amino acid backbone. The term also refers to nucleic acids containing modified bases.

“Nucleotide analogue” as used herein can be a modified deoxyribonucleoside; a modified ribonucleoside; a base-modified, sugar-modified, a phosphate-modified phosphate group, a phosphorothioate group, a phosphonate group, a methyl-phosphonate group, a phosphoramidate group, a formylacetyl group, a phosphorodithiorate group, a boranephosphate group, or a phosphotriester group.

The term “primer” or “nucleic acid probe” normally refers herein to an oligonucleotide used, for example in amplification of nucleic acids such as PCR. The primer can be comprised of unmodified and/or modified nucleotides, for example modified by a biotin group attached to the nucleotide at the 5′ end of the primer. The primer may contain at least 15 nucleotides, and preferably at least 18, 20, 22, 24 or 26 nucleotides.

The term “fragment” is intended to encompass a portion of a nucleotide or protein sequence. A nucleotide fragment may be at least about 15 contiguous nucleotides, preferably at least about 18, 20, 23 or 25 nucleotides, and can be 30, 40, 50, 100, 200 or more nucleotides in length. A protein fragment may be at least about 5 contiguous amino acids in length, preferably at least about 7, 10, 15, or 20 amino acids, and can be 25, 30, 40, 50 or more amino acids in length. A particularly useful protein fragment is one that retains activity, for example enzyme activity, cofactor binding capability, ability to bind other proteins, such as receptors, or ability to bind DNA. Another useful protein fragment is an isolated domain from a multidomain polypeptide. Such a fragment may thus retain activity residing in the particular domain.

The term “polypeptide” as used herein, refers to polymers of amino acids linked by peptide bonds and includes proteins, enzymes, peptides, and other gene products encoded by nucleic acids described herein.

The term “isolated” as used herein means that the material is removed from its original environment (e.g. the natural environment where the material is naturally occurring). For example, a polynucleotide or polypeptide while present in a living source organism is not isolated, but the same polynucleotide or polypeptide, which is separated from some or all of the coexisting materials in the natural system, is isolated. Such polynucleotides could for example be part of a vector and/or such polynucleotides or polypeptides could be part of a composition, and still be isolated in that the vector or composition is not part of the natural environment. When referring to a particular polypeptide, the term “isolated” refers to a preparation of the polypeptide outside its natural source and preferably substantially free of contaminants.

The term protein “domain” adopted herein is that of compactly folded structures with their own hydrophobic core. Different domains along a single polypeptide chain may act as independent units, to the extent that they can be excised from the chain, and still be shown to fold correctly, and may still exhibit biological activity such as the ability to catalyse a specific chemical reaction. Different domains within the same polypeptide may be more or less associated, such as only connected by a flexible linker region or tightly associated to appear as a single globular protein.

The term “homologous” used herein is defined as descending from a common ancestor, i.e. having the same evolutionary origin. Generally, homologous polypeptides are similar in appearance or structure, but not necessarily in function. Substantial sequence similarity, such as more than 30% sequence identity and/or conservation of characteristic amino acid residues, such as in defined or characteristic sequence motifs and/or functionally important residues, is indicative of homology. As used herein, homology is thus inferred from sequence comparison revealing substantial sequence similarity. “Sequence similarity” is suitably indicated by “0% sequence identity”.

“Thermostable” is defined herein as having the ability to withstand high temperatures above about 50° C. for at least 30 minutes without becoming irreversibly denatured. When referring to enzymes, the term thermostable indicates that the enzyme retains substantial enzymatic activity at temperatures above 50° C., such as at a desired temperature between 50° C. and 100° C. and preferably at a temperature in the range between 60° C.-100° C. Thermostable enzymes according to the present have optimal activity at a temperature above 40° C., such as in the range between 50° C. and 100° C., preferably at a temperature above about 60° C. such as above 65° C., or above 70° C. or above 75° C.

“Thermophilic bacteria”, also referred to as “thermophiles”, are defined as bacteria having optimum growth temperature above 50° C. “Thermophilic bacteriophages” or “thermostable bacteriophages” are defined as bacteriophages having thermophilic bacteria as hosts.

“Thermophilic isolate” as used herein refers to a bacterial isolate, which has been isolated from a high temperature environment and grown and maintained in a laboratory as a pure culture.

Methods of producing replicate copies of the same polynucleotide, such as PCR or gene cloning, are collectively referred to herein as “amplification” or “replication.” For example, single or double stranded DNA can be replicated to form another DNA with the same sequence. RNA can be replicated, for example, by RNA directed RNA polymerase, or by reverse transcribing the RNA and then performing a PCR. In the latter case, the amplified copy of the RNA is a DNA with the correlating or homologous sequence.

The polymerase chain reaction (“PCR”) is a reaction in which replicate copies are made of a target polynucleotide using one or more primers, and a catalyst of polymerization, such as a DNA polymerase, and particularly a thermally stable polymerase enzyme. Generally, PCR involves repeatedly performing a “cycle” of three steps: 1) “melting” in which the temperature is adjusted such that the DNA dissociates to single strands, 2) “annealing” in which the temperature is adjusted such that oligonucleotide primers are permitted to anneal to their complementary nucleotide sequence to form a duplex at one end of the polynucleotide segment to be amplified; and 3) “extension” or “synthesis” which can occur at the same or slightly higher and more optimum temperature than annealing, and during which oligonucleotides that have formed a duplex are elongated with a thermostable DNA polymerase. The cycle is then repeated until the desired amount of amplified polynucleotide is obtained. Methods for PCR amplification can be found in U.S. Pat. Nos. 4,683,195 and 4,683,202.

By “end-labelling” is meant that a suitable label, such as a radioactive label, is stably attached, typically covalently bonded, to one end of the ribonucleic acid, such as the 5′ terminal nucleotide of the nucleic acid. End-labelling according to the subject invention is accomplished by enzymatically attaching labelled chemical groups to the 5′ end of the nucleic acid, such that at least one labelled chemical group is present at the 5′ end of the end-labelled nucleic acid. By enzymatically attaching is meant that at least one labelled chemical group, such as a phosphate group, is attached to the 5′ terminal nucleotide of the nucleic acid with a thermostable polypeptide having 5′-kinase and/or 3′-phosphatase activity.

The labelled nucleotide employed in the subject methods is typically a modified adenine triphosphate, modified by incorporation of an atom or a chemical group providing a detectable signal. The detectable signal is typically a radioactive group, such as a phosphate group containing a radioactive isotope, such as P³² located in the gamma-phosphate group. Labels of interest are those that provide a detectable signal and do not substantially interfere with the ability of the labelled nucleotide to serve as a substrate for the thermostable polypeptide having 5′-kinase and/or 3′-phosphatase activity.

The methods disclosed herein involving the molecular manipulation of nucleic acids are known to those skilled in the art and are generally described e.g. in Ausubel, F. M. et al., “Short Protocols in Molecular Biology” John Wiley and Sons (1995); and Sambrook, J., et al., “Molecular Cloning, A Laboratory Manual” 2nd ed., Cold Spring Harbor Laboratory Press (1989).

As described herein, Applicants have isolated and characterized polypeptides having 5′-kinase and/or 3′-phosphatase activity.

The polypeptides of the invention show substantial 5′-kinase and/or 3′-phosphatase activity and are by inference substantially stable (i.e. correctly folded and soluble) at temperatures up to about 70° C. or higher. Preferred polypeptides of the invention retain at least 20% activity upon incubation for at least 24 hours at temperatures of at least about 60° C., and retain substantial activity at temperatures in the range from about 30° C. to about 70° C. This extended range of thermostability as compared to mesophilic counterparts is useful in various applications known to those skilled in the art and as set forth herein.

As outlined in Example 1, the applicants identified a putative gene product in the genome of bacteriophage RM378 that had a potential 5′-kinase activity and 3′-phophatase activity but the identity was still uncertain giving its unique sequence features and apparent domain arrangement. The amino acid sequence of the potential PNK gene product showed significant similarity only to the 5′ kinase domain of the PNK family characterized by T4 PNK and showed no significant similarity to the 3′ phosphatase domain in that family. Still, the similarity with the 5′ kinase domain was very low but they share the P-loop GXXXXGK(S/T) motif, which is characteristic for many phosphotransferase families. The 5′ kinase domain is located on the C-terminal end of the RM378 PNK in contrast to T4 PNK, suggesting that some kind of domain rearrangement has taken place, since many known polynucleotide kinases from both viral and eukaryotic origins have the same domain structure as in T4 PNK. (Amitsur, M. et al. (1987) Embo J 6, 2499-2503; Jilani, A. et al. (1999) J Cell Biochem 73, 188-203; Karimi-Busheri, F. et al., (1999) J Biol Chem 274, 24187-24194). T4 PNK and homologs previously found in other bacteriophages, including coliphage RB69, phage Aeh1 and very recently in mycobacteriophages Omega and Cjw1 and vibriophage KVP40, all have the kinase-phosphatase order of domains in contrast to the phosphatase-kinase order in mammalian PNKs (Zhu, H., et al. (2004) J Biol Chem, 18; 279(25):26358-69). Although distinctly different from both these groups by its unique phosphatase domain, RM378 PNK resembles the mammalian PNKs rather than other phage PNKs in terms of domain arrangement (Richardson, C. C. (1965) Proc Natl Acad Sci USA 54, 158-165; Zhu, H., et al. (2004) J Biol Chem, 18; 279(25):26358-69).

BLAST results reveal that the 3′ phosphohydrolase domain of RM378 PNK is related to the HD superfamily of phosphohydrolases, defined by the characteristic HD motif (Aravind, L., and Koonin, E. V. (1998) Trends Biochem Sci 23, 469-472), and distantly related to phosphodiesterase family (PDEs). The natural substrates of the PDEs are cyclic 3′,5′ AMP and GTP but these enzymes are often inhibited by some other phosphonucleotides, like adenosine-mono-phosphate and 2′,3′ cyclic mono-phosphate. Sequence analysis and BLAST searches showed that the RM378 PNK N-terminal domain shared similarity with poly(A) polymerases from eukaryotic origin. Poly(A) polymerases are responsible for mRNA adenylation, which is a control mechanism for RNA degradation in eubacteria. Poly(A) polymerases are of a nucleotidyl transferase (NTR) superfamily which includes CCA NTRs, poly(A) polymerase and DNA polymerase beta (Yue, D. et al. (1996) Rna 2, 895-908; Tomita, K., and Weiner, A. M. (2001) Science 294, 1334-1336; Tomita, K., and Weiner, A. M. (2002) J Biol Chem 277, 48192-48198). Two putative poly(A) polymerase like proteins from E. coli (accession no. AAN81695) and Clostridium acetobutylicum (accession no. NP_{—}347389) showed limited similarity to the whole RM378 PNK protein and shared similar size and domain orientation. These putative poly(A) polymerases shared both the 5′ kinase P-loop and the 3′ phosphohydrolase HD motifs, which suggests they might be related to the putative PNK gene product in bacteriophage RM378 and potentially had similar function. The overall similarity was low and the function of these putative poly(A) polymerases is unknown, therefore exact relationship cannot be resolved, without further investigation. The HD hydrolases are found together with a variety of other types of domains and display various domain architecture. Many of the proteins thus formed seem to have a function in nucleotide metabolism through a fusion of a HD domain to either a nucleotidyl transferase, helicase or a RNA binding domain (Aravind, L, and Koonin, E. V. (1998) Trends Biochem Sci 23, 469-472). The putative PNK gene product in bacteriophage RM378 would therefore be another example of this theme. As outlined in examples 2 and 3, the applicants have demonstrated after cloning and expression of the putative PNK gene that the gene product does possess 5′-kinase and 3′-phosphatase activity.

The RM378 polynucleotide kinase is a bifunctional enzyme postulated to catalyse the same or very similar reactions as T4 polynucleotide kinase. T4 PNK heals nicked tRNA molecules after cleavage with the ACNase in prr+ E. coli strains, and therefore overcomes the ACNase suicidal mechanism, which has the purpose of limiting the T4 phage infection, and is an interesting example of altruistic behaviour among bacteria (Amitsur, M. et al., (1987) Embo J 6, 2499-2503; Sirotkin, K. et al., (1978) J Mol Biol 123, 221-233). Our studies suggest that the phage RM378 needs to counter similar RNA degradation mechanisms in R. marinus. This observation is based on the fact that the bacteriophage RM378 seems to be armed with both RNA ligase (Blondal, T. et al., (2003) Nucleic Acids Res 31, 7247-7254) and polynucleotide kinase.

As PNK and RNA ligase are two components with common purpose and presumably acting in conjunction, it is not surprising to find the corresponding genes located close to each other in the genome of bacteriophage T4, separated by about 1400 bp, and running in the same direction. It may seem logical to expect similar organization in the genome of bacteriophage RM378 although overall general organisation of the two phage genomes is not similar judged from identified homologous genes (Hjörleifsdottir, S. H. et al., (2002) U.S. Pat. No. 6,492,161). As described in Example 1, efforts to locate the PNK gene after the discovery of the RNA ligase gene did not identify a likely candidate ORF in the immediate vicinity of the RNA ligase gene. A PNK gene was finally located still relatively close, about 6400 bp downstream, but running in the opposite direction.

Characterization of the RM378 PNK, described in Examples 2 and 3, showed that it is a moderately thermostable protein with an apparent temperature optimum in the range of 65-70° C. This was not unexpected since R. marinus natural environment is of similar temperature, with optimum growth conditions about 65° C. (Alfredsson, G. A. et al., (1988) J. Gen. Microbiol. 134, 299-306). The PNK shows high activity on both RNA and DNA and similar activity for single- and double-stranded DNA. The 5′ kinase activity is dependent upon a divalent cation, were both Mg2+ and Mn2+ work equally well. As previously described for other enzymes including nucleotidyl transferase enzymes like T4 PNK, and T4 DNA and RNA ligase, the RM378 PNK shows a multi-fold increase in kinase activity in the presence of 5-10% PEG6000 (Tessier, D. C., Brousseau, R., and Vemet, T. (1986) Anal Biochem 158, 171-178; Pheiffer, B. H., and Zimmerman, S. B. (1983) Nucleic Acids Res 11, 7853-7871; Harrison, B., and Zimmerman, S. B. (1986) Anal Biochem 158, 307-315; Harrison, B., and Zimmerman, S. B. (1986) Nucleic Acids Res 14, 1863-1870). Also as shown for T4 PNK, RM378 PNK shows similar inhibition when presented with ADP in the 5′ reaction resulting close to complete inhibition of the kinase reaction. When only ADP and 5′ phosphorylated oligomer were present in the reaction mixture, the oligomer was dephosphorylated, showing that the reaction can readily go in both directions, as has also been observed with T4 PNK (Lillehaug, I. R. (1978) Biochim Biophys Acta 525, 357-363; Lillehaug, J. R. (1977) Eur J Biochem 73, 499-506; van de Sande, I. H. et al., (1973) Biochemistry 12, 5050-5055). When a mixture of ATP, ADP and a 5′ hydroxylated oligomer is used, increasing concentration of ADP inhibits the 5′ kinase reaction with a complete inhibition when in 1:5 molar excess suggesting that the two components are competing for the active site of the kinase domain. On the other hand, the exchange reaction did not seem to be affected by increasing ADP concentration, constantly giving 3-8% exchange on 5′ phosphorylated oligomers, even when no ADP was present in the exchange reaction. This exchange activity is relatively low and the ADP independence may be caused by i) substrate independent conversion of ATP to ADP and Pi as seen with T4 PNK (Galburt, E. A. et al., (2002) Structure (Camb) 10, 1249-1260) or ii) that the high temperature causes some minor degradation of the oligomers 5′ phosphate group, causing it to be re-phosphorylated and in the process generating free ADP, starting the exchange reaction. At higher ADP concentration the inhibitory effect of ADP limits the reaction.

The structure of T4 PNK reveals a narrow entrance to the kinase active site (Wang, L. K. et al., (2002) Embo J 21, 3873-3880). Modelling of substrate binding suggest that only single-stranded nucleic add substrate can be accommodated at the binding site and thus providing a possible explanation of the relatively lower efficiency of the enzyme catalysed phosphorylation using blunt end double stranded DNA substrates. An enzyme working at higher temperatures, where strand separation in double-stranded nucleic acids is facilitated, is therefore expected to be much more efficient for phosphorylation of double-stranded DNA with blunt-ends or 3′-overhangs.

The 3′ phosphohydrolase activity has somewhat different physical properties when compared to the kinase activity characteristics. The first striking observation was that the pH optimum of the phosphatase activity is around pH 6 compared to pH 8-9 for the kinase activity. This may suggest that the kinase and phosphohydrolase activity reside in different protein domains, which is in line with biochemical and structural observations for T4 PNK (Wang, L. K. et al., (2002) Embo J 21, 3873-3880; Wang, L. K., and Shuman, S. (2001) J Biol Chem 276, 26868-26874; Galburt, E. A. et al., (2002) Structure (Camb) 10, 1249-1260; Wang, L. K., and Shuman, S. (2002) Nucleic Acids Res 30, 1073-1080). Another difference is the preference for Mn²⁺ over Mg²⁺, resulting in much more phosphohydrolase activity in the presence of Mn⁺² than in the presence of Mg²⁺. Still the enzyme showed some activity on CAMP in the presence of only Mg²⁺, meaning that the enzyme could most likely perform its function in the presence of Mg²⁺ which is much more abundant in the cell compared to Mn²⁺. Compared to the 3′phospahtase activity of T4 PNK, the polypeptides provided by the present invention have distinctly different substrate specificity and may thus be more suitable to certain applications than T4 PNK. This may for example include procedures where a lower 3′phosphatase activity on oligonucleotides is desired.

The RM378 PNK provides an example of domain reconstruction, were homologous tRNA repair systems have changed over time. When comparing the three complete virus derived tRNA repair systems known today, it becomes clear how different solutions could be applied to solve the tRNA degradation problem. The T4 is armed with a RNA ligase and a PNK (David, M. et al., (1982) Virology 123, 480-483); and although RM378 has a homologous RNA ligase (Blondal, T. et al., (2003) Nucleic Acids Res 31, 7247-7254), one of the two domains in RM378 PNK is replaced with an analogous but apparently non-homologous domain compared to T4 PNK, and the orientation of the domains is reversed, resulting in similar activity but adapted to elevated temperature. The ACNV provides the third known solution where the two polypeptides are merged into one multidomain protein with the same basic functions as the two above (Martins, A., and Shuman, S. (2004) J Biol Chem, in press).

The discovery of a thermostable PNK and the characterization of its activities makes possible further applications for the manipulation of nucleic acids. Applications utilizing T4 PNK indicate the utility of other enzymes having similar activities. The present invention provides characterization of a PNK with activities generally comparable to that of T4 PNK but distinctly different properties, thus broadening the scope of applications using enzymes of this type.

The invention pertains to the use of the polypeptides provided by the invention in various applications including nucleotide labelling, oligonucleotide synthesis and gene synthesis.

The invention pertains to a method of transferring a phosphate group or phosphate analogues from nucleotide triphosphate or nucleotide analogues triphosphates to 5′ ends of nucleic acids or nucleic acid analogues using a isolated thermostable polypeptides having 5′-kinase and/or 3′-phosphatase activity. In certain embodiments, the thermostable polypeptides having 5′-kinase and/or 3′-phosphatase activity can be derived from a thermostable bacteriophage; the nucleic adds can be RNA or DNA; the RNA or DNA can be single stranded; and the nucleotide analogues may contain modified bases, modified sugars and/or modified phosphate groups.

In yet another embodiment, a method of synthesizing an oligonucleotide polymer by repeating cycles of combining a primer oligonucleotide and a blocked oligonucleotide is described, comprising: a) combining the primer oligonucleotide and an oligonucleotide blocked at the 3′ or 5′ end in the presence of a RNA ligase, thereby forming an extended primer with a blocked 3′ or 5′ end; b) removing the blocked phosphate group at the 3′ or 5′ end or adding a phosphate group to the 5′ end of the extended primer using thermostable polypeptides having 5′-kinase and/or 3′-phosphatase activity; and c) repeating a) and b) using the extended primer from b) as the primer for a) wherein an oligonucleotide polymer is formed. In certain embodiments, the formed oligonucleotide polymer comprises a gene or a part of a gene coding for a polypeptide.

Methods are provided for end-labelling nucleic acids with suitable labels such as radioactively labels. In one embodiment, nucleic acid is contacted with radioactively labelled nucleotides, e.g. ribonucleotide triphosphate containing a radioactive P³² atom, in the presence of thermostable polypeptides having 5′-kinase activity under conditions sufficient for covalent attachment of one or more of the labelled atoms to the 5′ end of the nucleic acid to occur.

A polypeptide having 5′-kinase and/or 3′-phosphatase activity is often used in various applications for modifications of nucleic acids prior to other modifications of nucleic acids using polypeptides with other enzymatic activities. Phosphorylation of the 5′ end of a nucleic acid is often prerequisite for subsequent modifications such as ligation to another nucleic acid. Labelling of nucleic acids is also often required before using other enzymes. A polypeptide having 5′-kinase and/or 3′-phosphatase activity is thus often used in same applications as other enzymes. Examples of enzymes of this type are RNA ligases, DNA ligases, exonucleases, DNA polymerases, RNA polymerases and phosphatases. A thermostable polypeptide having 5′-kinase and/or 3′-phosphatase activity provided by the invention can suitably be used in combination with such enzymes as well as other components in kits for various applications.

Also provided are kits for use in practicing the methods of the subject invention. The subject kits typically include at least a thermostable polypeptide having 5′-kinase and/or 3′-phosphatase activity, as described above, and a suitable reaction buffer. The kit may also include nucleotides, e.g. nucleotide triphosphate such as ATP, labelled or unlabelled. The subject kits may further include additional reagents necessary and/or desirable for use in practicing the subject methods, where additional reagents of interest include: an aqueous buffer medium (either prepared or present in its constituent components, where one or more of the components may be premixed or all of the components may be separate); RNase inhibitors, control substrates, control nucleic acids, and the like. The subject kits may also include other polypeptides having various other enzymatic activities. These activities include but are not limited to ligase activity, polymerase activity and nuclease activity and other activities of polypeptides having enzymatic activity on nucleic acids. Examples of enzymes having those activities are RNA ligases, DNA ligases, exonucleases, DNA polymerases, RNA polymerases and phosphatases. The various reagent components of the kits may be present in separated containers, or may all be pre-combined into a reagent mixture for combination with to be labelled ribonucleic acid. A set of instructions will also typically be included, where the instructions may be associated with a package insert and/or the packaging of the kit or the components thereof.

The kits of the present invention typically will include buffering components and may come in a ready-to-use aqueous solution or in a dry formulation (e.g. lyophilized) optionally comprising buffer components to obtain a suitable buffered solution upon addition of water.

A non-limiting example of a kit provided by the invention is a kit containing an isolated thermostable polypeptide having 5′-kinase activity and an isolated heat-labile alkaline phosphatase. Alkaline phosphatases, are known in the prior art and are useful for example for removal of 5′phosphate groups on nucleic acids prior to labelling with an enzyme having 5′-kinase activity. Heat-labile alkaline phosphatases, such as shrimp alkaline phosphatase (Olsen, R. L., Øverbø, K. and Myrnes, B. (1991): Comp. Biochem. Physiol. 99B: 755-761), are especially useful as these enzymes can be inactivated by heat treatment prior to addition of kinase and do not require more cumbersome methods of removal of phosphatase activity, for example with phenol extraction. The present invention provides a heat-stable polypeptide having 5′-kinase activity which can be used in kits together with heat-labile alkaline phosphatases and provides the additional advantage of not having to lower the temperature again and add the kinase afterwards, after heat treatment, but instead having a kit with both enzymes present in the same mixture and simultaneously inactivate the alkaline phosphatase and activate the 5′-kinase. A method for labelling of nucleic acids can be conveniently performed with such a kit requiring only a first incubation at a relatively low temperature, where the alkaline phosphatase is active, followed by second incubation at higher temperature, such as 65° C., whereupon the heat-labile alkaline phosphatase is inactivated and the thermostable kinase is activated.

### Nucleic Acids of the Invention

One aspect of the invention pertains to isolated nucleic acid sequences, encoding a polypeptide having 5′-kinase and/or 3′-phosphatase activity. A nucleic acid sequence of an isolated nucleic acid of the invention is shown in FIG. 1 (SEQ ID NO: 1).

The nucleic acid molecules of the invention can be DNA or RNA molecules, for example, mRNA. DNA molecules can be double-stranded or single-stranded; single stranded RNA or DNA can be the coding, or sense, strand or the non-coding, or antisense, strand. Preferably, the nucleic acid molecule comprises at least about 100 nucleotides, more preferably at least about 150 nucleotides, and even more preferably at least about 200 nucleotides. In one embodiment the nucleotide sequence is one that encodes at least a fragment of the amino acid sequence of a polypeptide; alternatively, the nucleotide sequence can include at least a fragment of a coding sequence along with additional non-coding sequences such as non-coding 3′ and 5′ sequences (including regulatory sequences, for example).

Additionally, the nucleotide sequence(s) can be fused to a marker sequence, for example, a sequence which encodes a polypeptide to assist in isolation or purification of the polypeptide. Representative sequences include, but are not limited to, those which encode a glutathione-S-transferase (GST) fusion protein or a histidine tag. In one embodiment, the nucleotide sequence contains a single ORF in its entirety (e.g., encoding a polypeptide, as described below); or contains a nucleotide sequence encoding an active derivative or active fragment of the polypeptide; or encodes a polypeptide which has substantial sequence identity to the polypeptides described herein.

The nucleic acid molecule can be fused to other coding or regulatory sequences. Thus, recombinant DNA contained in a vector is included in the definition of “isolated” as used herein. Also, isolated nucleic acid molecules include recombinant DNA molecules in heterologous host cells, as well as partially or substantially purified DNA molecules in solution. “Isolated” nucleic acid molecules also encompass in vivo and in vitro RNA transcripts of the DNA molecules of the present invention. An isolated nucleic acid molecule or nucleotide sequence can include a nucleic acid molecule or nucleotide sequence which is synthesized chemically or by recombinant means. Therefore, recombinant DNA contained in a vector is included in the definition of “isolated” as used herein. Also, isolated nucleotide sequences include recombinant DNA molecules in heterologous organisms, as well as partially or substantially purified DNA molecules in solution. In vivo and in vitro RNA transcripts of the DNA molecules of the present invention are also encompassed by “isolated” nucleotide sequences. Such isolated nucleotide sequences are useful in the manufacture of the encoded polypeptide, as probes for isolating homologous sequences, for gene mapping or for detecting expression of the gene, such as by Northern blot analysis.

The present invention also pertains to nucleotide sequences which are not necessarily found in nature but which encode the polypeptides of the invention. Thus, DNA molecules which comprise a sequence which is different from the naturally occurring nucleotide sequence but which, due to the degeneracy of the genetic code, encode the polypeptides of the present invention, are the subject of this invention. The invention also encompasses variations of the nucleotide sequences of the invention, such as those encoding active fragments or active derivatives of the polypeptides as described below. Such variations can be naturally occurring, or non-naturally occurring, such as those induced by various mutagens and mutagenic processes. Intended variations include, but are not limited to, addition, deletion and substitution of one or more nucleotides which can result in conservative or non-conservative amino acid changes, including additions and deletions. Preferably, the nucleotide or amino acid variations are silent or conservative; that is, they do not alter the characteristics (e.g. structure, flexibility and electrostatic microenvironment within the protein) or activity of the encoded polypeptide. However, variations may alter the various properties of the polypeptides encoded by the nucleic acids while preferably still retaining substantial 5′-kinase and/or 3′-phosphatase activity.

The invention described herein also relates to fragments of the isolated nucleic acid molecules described herein. The term “fragment” is intended to encompass a portion of a nucleotide sequence described herein which is from at least about 15 contiguous nucleotides to at least about 50 contiguous nucleotides or longer in length; such fragments are useful as probes and also as primers. Particularly preferred primers and probes selectively hybridize to the nucleic acid molecule encoding the polypeptides described herein. For example, fragments which encode polypeptides that retain enzyme activity, as described below, are particularly useful.

Other alterations of the nucleic acid molecules of the invention can include, for example, labeling, methylation, internucleotide modifications such as uncharged linkages (e.g., methyl phosphonates, phosphotriesters, phosphoamidates, carbamates), charged linkages (e.g., phosphorothioates, phosphorodithioates), pendent moieties (e.g., polypeptides), intercalators (e.g., acridine, psoralen), chelators, alkylators, and modified linkages (e.g., alpha anomeric nucleic acids). Also included are synthetic molecules that mimic nucleic acid molecules in the ability to bind to designated sequences via hydrogen bonding and other chemical interactions. Such molecules include, for example, those in which peptide linkages substitute for phosphate linkages in the backbone of the molecule (polypeptide nucleic acids, as described in Nielsen, et al., Science, 254:1497-1500 (1991)).

The invention also encompasses nucleic acid molecules which hybridize under high stringency hybridization conditions, such as for selective hybridization, to a nucleotide sequence described herein (e.g., nucleic acid molecules which specifically hybridize to a nucleotide sequence encoding polypeptides described herein, and, optionally, have an activity of the polypeptide). Hybridization probes are oligonucleotides which bind in a base-specific manner to a complementary strand of nucleic acid.

Such nucleic acid molecules can be detected and/or isolated by specific hybridization (e.g., under high stringency conditions). “Stringency conditions” for hybridization is a term of art which refers to the incubation and wash conditions, e.g., conditions of temperature and buffer concentration, which permit hybridization of a particular nucleic acid to a second nucleic acid; the first nucleic acid may be perfectly (i.e., 100%) complementary to the second, or the first and second may share some degree of complementarity which is less than perfect (e.g., 60%, 75%, 85%, 95%). For example, certain high stringency conditions can be used which distinguish perfectly complementary nucleic acids from those of less complementarity.

“High stringency conditions”, “moderate stringency conditions” and “low stringency conditions” for nucleic acid hybridizations are explained on pages 2.10.1-2.10.16 and pages 6.3.1-6 in Current Protocols in Molecular Biology (Ausubel, F. M. et al., “Current Protocols in Molecular Biology”, John Wiley & Sons, (2001)) the teachings of which are hereby incorporated by reference. The exact conditions which determine the stringency of hybridization depend not only on ionic strength (e.g., 0.2×SSC, 0.1×SSC), temperature (e.g., room temperature, 42° C., 68° C.) and the concentration of destabilizing agents such as formamide or denaturing agents such as SDS, but also on factors such as the length of the nucleic acid sequence, base composition, percent mismatch between hybridizing sequences and the frequency of occurrence of subsets of that sequence within other non-identical sequences. Thus, high, moderate or low stringency conditions can be determined empirically.

By varying hybridization conditions from a level of stringency at which no hybridization occurs to a level at which hybridization is first observed, conditions which will allow a given sequence to hybridize (e.g., selectively) with the most similar sequences in the sample can be determined.

Exemplary conditions are described in Krause, M. H. and S. A. Aaronson, Methods in Enzymology, 200:546-556 (1991). Also, in, Ausubel, et al., “Current Protocols in Molecular Biology”, John Wiley & Sons (2001), which describes the determination of washing conditions for moderate or low stringency conditions. Washing is the step in which conditions are usually set so as to determine a minimum level of complementarity of the hybrids. Generally, starting from the lowest temperature at which only homologous hybridization occurs, each degree C. by which the final wash temperature is reduced (holding SSC concentration constant) allows an increase by 1% in the maximum extent of mismatching among the sequences that hybridize. Generally, doubling the concentration of SSC results in an increase in Tm of 17° C. Using these guidelines, the washing temperature can be determined empirically for high, moderate or low stringency, depending on the level of mismatch sought.

For example, a low stringency wash can comprise washing in a solution containing 0.2×SSC/0.1% SDS for 10 minutes at room temperature; a moderate stringency wash can comprise washing in a pre-warmed solution (42° C.) solution containing 0.2×SSC/0.1% SDS for 15 min at 42° C.; and a high stringency wash can comprise washing in pre-warmed (68° C.) solution containing 0.1×SSC/0.1% SDS for 15 min at 68° C. Furthermore, washes can be performed repeatedly or sequentially to obtain a desired result as known in the art.

Equivalent conditions can be determined by varying one or more of the parameters given as an example, as known in the art, while maintaining a similar degree of identity or similarity between the target nucleic acid molecule and the primer or probe used. Hybridizable nucleic acid molecules are useful as probes and primers, e.g., for diagnostic applications.

Such hybridizable nucleotide sequences are useful as probes and primers for diagnostic applications. As used herein, the term “primer” refers to a single-stranded oligonucleotide which acts as a point of initiation of template-directed DNA synthesis under appropriate conditions (e.g., in the presence of four different nucleoside triphosphates and an agent for polymerization, such as, DNA or RNA polymerase or reverse transcriptase) in an appropriate buffer and at a suitable temperature. The appropriate length of a primer depends on the intended use of the primer, but typically ranges from 15 to 30 nucleotides. Short primer molecules generally require cooler temperatures to form sufficiently stable hybrid complexes with the template. A primer need not reflect the exact sequence of the template, but must be sufficiently complementary to hybridize with a template. The term “primer site” refers to the area of the target DNA to which a primer hybridizes. The term “primer pair” refers to a set of primers including a 5′ (upstream) primer that hybridizes with the 5′ end of the DNA sequence to be amplified and a 3′ (downstream) primer that hybridizes with the complement of the 3′ end of the sequence to be amplified.

The invention also pertains to nucleotide sequences which have a substantial identity with the nucleotide sequences described herein; particularly preferred are nucleotide sequences which have at least about 10%, preferably at least about 20%, more preferably at least about 30%, more preferably at least about 40%, even more preferably at least about 50%, yet more preferably at least about 70%, still more preferably at least about 80%, and even more preferably at least about 90% identity, and still more preferably 95% identity, with nucleotide sequences described herein. Particularly preferred in this instance are nucleotide sequences encoding polypeptides having 5′-kinase and/or 3′-phosphatase activity as described herein.

To determine the percent identity of two nucleotide sequences, the sequences are aligned for optimal comparison purposes (e.g., gaps can be introduced in the sequence of a first nucleotide sequence). The nucleotides at corresponding nucleotide positions are then compared. When a position in the first sequence is occupied by the same nucleotide as the corresponding position in the second sequence, then the molecules are identical at that position. The determination of percent identity or similarity scores between two sequences can be accomplished using a mathematical algorithm. A preferred, non-limiting example of a mathematical algorithm utilized for the comparison of two sequences is the algorithm of Karlin, et al., Proc. Natl. Acad. Sci. USA, 90:5873-5877 (1993). Such an algorithm is incorporated into the BLAST programs (e.g. BLASTN for nucleotide sequences or BLASTP for protein sequences) which can be used to identify sequences with high similarity scores to nucleotide or protein sequences of the invention. To obtain gapped alignments for comparison purposes, Gapped BLAST can be utilized as described in Altschul et al., Nucleic Acids Res, 25:3389-3402 (1997). When utilizing BLAST and Gapped BLAST programs, the default parameters of the respective programs (e.g., BLASTN) can be used. See the BLAST programs provided by National Center for Biotechnology Information, National Library of Medicine, National Institutes of Health. In one embodiment, parameters for sequence comparison can be set at W=12. Parameters can also be varied (e.g., W=5 or W=20). The value “W” determines how many continuous nucleotides must be identical for the program to identify two sequences as containing regions of identity.

Alignment of sequences and calculation of sequence identity may also be done using for example the Needleman and Wunsch global alignment algorithm (Needleman, S. B. and Wunsch, C. D. (1970) J. Mol. Biol. 48, 443-453) useful for both protein and DNA alignments and discussed further below.

The invention also provides expression vectors containing a nucleic acid sequence encoding a polypeptide described herein (or an active derivative or fragment thereof), operably linked to at least one regulatory sequence. Many expression vectors are commercially available, and other suitable vectors can be readily prepared by the skilled artisan. “Operably linked” is intended to mean that the nucleotide sequence is linked to a regulatory sequence in a manner which allows expression of the nucleic acid sequence. Regulatory sequences are art-recognized and are selected to produce the polypeptide or active derivative or fragment thereof. Accordingly, the term “regulatory sequence” includes promoters, enhancers, and other expression control elements which are described in Goeddel, Gene Expression Technology: Methods in Enzymology 185, Academic Press, San Diego, Calif. (1990). For example, the native regulatory sequences or regulatory sequences native to organism can be employed. It should be understood that the design of the expression vector may depend on such factors as the choice of the host cell to be transformed and/or the type of polypeptide desired to be expressed. For instance, the polypeptides of the present invention can be produced by ligating the cloned gene, or a portion thereof, into a vector suitable for expression in an appropriate host cell (see, for example, Broach, et al., Experimental Manipulation of Gene Expression, ed. M. Inouye (Academic Press, 1983) p. 83; Molecular Cloning: A Laboratory Manual, 2nd Ed., ed. Sambrook et al. (Cold Spring Harbor Laboratory Press, 1989) Chapters 16 and 17). Typically, expression constructs will contain one or more selectable markers, including, but not limited to, the gene that encodes dihydrofolate reductase and the genes that confer resistance to neomycin, tetracycline, ampicillin, chloramphenicol, kanamycin and streptomycin resistance. Thus, prokaryotic and eukaryotic host cells transformed by the described expression vectors are also provided by this invention. For instance, cells which can be transformed with the vectors of the present invention include, but are not limited to, bacterial cells such as Thermus scotoductus, Thermus thermophilus, E. coli (e.g., E. coli K12 strains), Streptomyces, Pseudomonas, Bacillus, Serratia marcescens and Salmonella typhimurium. The host cells can be transformed by the described vectors by various methods (e.g., electroporation, transfection using calcium chloride, rubidium chloride, calcium phosphate, DEAE-dextran, or other substances; microprojectile bombardment; lipofection, infection where the vector is an infectious agent such as a retroviral genome, and other methods), depending on the type of cellular host. The nucleic acid molecules of the present invention can be produced, for example, by replication in such a host cell, as described above. Alternatively, the nucleic acid molecules can also be produced by chemical synthesis.

The isolated nucleic acid molecules and vectors of the invention are useful in the manufacture of the encoded polypeptide, as probes for isolating homologous sequences (e.g., from other spedes), as well as for detecting the presence of a DNA construct comprising a nucleic add sequence of the invention in a culture of host cells.

The nucleotide sequences of the nucleic add molecules described herein (e.g., a nucleic acid molecule comprising SEQ ID NO: 1 as shown in FIG. 1, such as a nucleic acid molecule comprising the open reading frames can be amplified by methods known in the art. For example, this can be accomplished by e.g., PCR. See generally PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. No. 4,683,202.

Other suitable amplification methods include the ligase chain reaction (LCR) (see Wu and Wallace, Genomics, 4:560 (1989), Landegren, et al., Science, 241:1077 (1988), transcription amplification (Kwoh, et al., Proc. Natl. Acad. Sci. USA 86:1173 (1989)), and self-sustained sequence replication (Guatelli, et al., Proc. Nat. Acad. Sci. USA, 87:1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

The amplified DNA can be radioactively labeled and used as a probe for screening a library or other suitable vector to identify homologous nucleotide sequences. Corresponding clones can be isolated, DNA can be obtained following in vivo excision, and the cloned insert can be sequenced in either or both orientations by art recognized methods, to identify the correct reading frame encoding a protein of the appropriate molecular weight. For example, the direct analysis of the nucleotide sequence of homologous nucleic acid molecules of the present invention can be accomplished using either the dideoxy chain termination method or the Maxam-Gilbert method (see Sambrook et al., Molecular Cloning, A Laboratory Manual (2nd Ed., CSHP, New York 1989); Zyskind et al., Recombinant DNA Laboratory Manual, (Acad. Press, 1988)). Using these or similar methods, the protein(s) and the DNA encoding the protein can be isolated, sequenced and further characterized.

### Polypeptides of the Invention

The invention additionally relates to isolated thermostable polypeptides with 5′-kinase and/or 3′-phosphatase activity.

For the purpose of the present invention, “polypeptides having 5′-kinase and/or 3′-phosphatase activity” is defined as described above. Briefly, a polypeptide having a 5′-kinase activity catalyzes the transfer of the gamma-phosphate of a nucleoside 5′-triphosphate to the 5′-hydroxyl terminus of a nucleic acid. A polypeptide having a 3′-phosphatase activity catalyzes the hydrolysis of 3′ phosphoryl groups on nucleic acids. 5′-kinase and 3′-phosphatase activities can be assayed individually with suitable assays as described above and also in the Examples.

The present invention provides an isolated thermostable polypeptide having 5′-kinase and 3′-phosphatase activity and isolated nucleic acids of the corresponding gene. As described in the Examples herein, the applicants have cloned the gene and expressed and characterized the corresponding recombinant polypeptide having 5′-kinase and 3′-phosphatase activity.

The preferred isolated polypeptides provided by the invention preferably have two different enzymatic activities, a 5′-kinase activity and a 3′-phosphatase activity. These activities have different pH activity range, about pH 8.5 for the 5′-kinase activity and about pH 6 for the phosphatase activity, but similar temperature optima in the range of at about 70-75° C.

It will be appreciated that the invention provides polypeptides having 5′-kinase activity that comprise a sequence substantially similar to the kinase domain of polypeptides of the invention that have both a 5′-kinase and a 3′-phosphatase domain. Such 5′-kinase active polypeptides may have, e.g., an amino add sequence substantially similar to the sequence of residues 174-350 of SEQ ID NO: 2 or an 5′-kinase active fragment thereof, e.g. where terminal residues that are not necessary for correct folding and function have been eliminated, such as, e.g., residues 186-340 of SEQ ID NO: 2. Likewise, the invention provides polypeptides having 3′-phosphatase activity that comprise a sequence substantially similar to residues 1-178 or 3′-phosphatase active fragments thereof, for example, where terminal residues not necessary for correct folding and function are eliminated.

In one aspect, the present invention relates to polypeptides having 5′-kinase and/or 3′-phosphatase activity with a temperature optimum of at least 60° C., preferably the temperature optimum is in the range 60° C. to 120° C., more preferably in the range 60° C. to 100° C., even more preferably in the range of 60° C. to 80° C. and most preferably in the range of 65° C. to 75° C.

In one embodiment the invention relates to isolated polypeptides having optimum 5′-kinase and/or 3′-phosphatase activity preferably in the range of about pH 5 to pH 9.

The polypeptides of the invention can be partially or substantially purified (e.g., purified to homogeneity), and/or are substantially free of other polypeptides. According to the invention, the amino acid sequence of the polypeptide can be that of the naturally occurring polypeptide or can comprise alterations therein. Polypeptides comprising alterations are referred to herein as “derivatives” of the native polypeptide. Such alterations include conservative or non-conservative amino acid substitutions, additions and deletions of one or more amino acids; however, such alterations should preserve the 5′-kinase and/or 3′-phosphatase activity of the polypeptide, i.e., the altered or mutant polypeptides of the invention are active derivatives of the naturally occurring polypeptide having 5′-kinase and/or 3′-phosphatase activity. Preferably, the amino acid substitutions are of minor nature, i.e. conservative amino acid substitutions that do not significantly alter the folding or activity of the polypeptide. Deletions are preferably small deletions, typically of one to 30 amino acids. Additions are preferably small amino- or carboxy-terminal extensions, such as amino-terminal methionine residue; a small linker peptide of up to about 25 residues; or a small extension that facilitates purification by changing net charge or another function, such as a poly-histidine tail, an antigenic epitope or a binding domain. The alteration(s) preferably preserve the three dimensional configuration of the active site of the native polypeptide, or can preferably preserve the activity of the polypeptide (e.g. any mutations preferably preserve the ability of the polypeptides of the present invention to catalyze the transfer of the gamma-phosphate of a nucleoside 5′-triphosphate to the 5′-hydroxyl terminus of a nucleic acid and/or the ability to catalyze the hydrolysis of 3′phosphoryl groups on nucleic acids. (Richardson C. C. (1981), in The enzymes vol XIV, P. D. Boyer, ed. Volume 14 (Academic Press, San Diego), pp. 299-314). The presence or absence of activity or activities of the polypeptide can be determined by various standard functional assays including, but not limited to, assays for binding activity or enzymatic activity.

Additionally included in the invention are active fragments of the polypeptides described herein, as well as fragments of the active derivatives described above. An “active fragment” as referred to herein, is a portion of polypeptide (or a portion of an active derivative) that retains the polypeptide's 5′-kinase and/or 3′-phosphatase activity, as described above. Appropriate amino acid alterations can be made on the basis of several criteria, including hydrophobicity, basic or acidic character, charge, polarity, size of side chain, the presence or absence of a functional group (e.g., —SH or a glycosylation site), and aromatic character. Assignment of various amino acids to similar groups based on the properties above will be readily apparent to the skilled artisan; further appropriate amino acid changes can also be found in Bowie, et al., Science, 247:1306-1310 (1990). For example, conservative amino acid replacements can be those that take place within a family of amino acids that are related in their side chains. Genetically encoded amino acids are generally divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. Phenylalanine, tryptophan and tyrosine are sometimes classified jointly as aromatic amino acids. For example, it is reasonable to expect that an isolated replacement of a leucine with an isoleucine or valine, an aspartate with a glutamate, a threonine with a serine or a similar conservative replacement of an amino acid with a structurally related amino acid will not have a major effect on activity or functionality.

In one embodiment the polypeptides of the invention are fusion polypeptides comprising all or a portion (e.g., an active fragment) of an amino acid sequence of the invention fused to an additional component, with optional linker sequences. Additional components, such as radioisotopes and antigenic tags, can be selected to assist in the isolation or purification of the polypeptide or to extend the half-life of the polypeptide; for example, a hexahistidine tag would permit ready purification by nickel chromatography. The fusion protein can contain, e.g., a glutathione-S-transferase (GST), thioredoxin (TRX) or maltose binding protein (MBP) component to facilitate purification; kits for expression and purification of such fusion proteins are commercially available. The polypeptides of the invention can also be tagged with an epitope and subsequently purified using antibody specific to the epitope using art recognized methods. Additionally, all or a portion of the polypeptide can be fused to carrier molecules, such as immunoglobulins, for many purposes, including increasing the valency of protein binding sites. For example, the polypeptide or a portion thereof can be linked to the Fc portion of an immunoglobulin; for example, such a fusion could be to the Fc portion of an IgG molecule to create a bivalent form of the protein.

Also encompassed by the invention are polypeptides having 5′-kinase and/or 3′-phosphatase activity which have at least about 30% sequence identity (i.e., polypeptides which have substantial sequence identity) to the amino acid sequence of SEQ ID NO: 2 described herein but preferably higher sequence identity, such as at least about 50% or about 60% sequence identity and more preferable at least about 70% or about 75% sequence identity, and more preferably at least about 80% or at least 90% sequence identity such as at least about 95% or 97% sequence identity such as at least about 99% sequence identity to said sequence of SEQ ID NO: 2. However, polypeptides exhibiting lower levels of overall sequence identity are also useful, particular if they exhibit higher identity over one or more particular domains of the polypeptide. For example, polypeptides sharing high degrees of identity over domains or characteristic sequence motifs necessary for particular activities, such as binding or enzymatic activity, are included herein.

Algorithms for sequence comparisons and calculation of “sequence identity” are known in the art as discussed above, such as BLAST, described in Altschul et al., J. Mol. Biol. (1990) 215:403-10 or the Needleman and Wunsch algorithm (Needleman, S. B. and Wunsch, C. D. (1970) J. Mol. Biol. 48, 443-453). Generally, the default settings with respect to e.g. “scoring matrix” and “gap penalty” will be used for alignment. The percentage sequence identity values referred to herein refer to values as calculated with the Needleman and Wunsch algorithm such as implemented in the program Needle (Rice, P. Longden, I. and Bleasby, A. “EMBOSS: The European Molecular Biology Open Software Suite” Trends in Genetics June 2000, vol 16, No 6. pp. 276-277) using the default scoring matrix EBLOSUM62 for protein sequences, (or scoring matrix EDNAFULL for nucleotide sequences) with opening gap penalty set to 10.0 and gap extension penalty set to 0.5. The sequence identity is thus the percentage of identical matches between the two sequences over the aligned region including any gaps in the length.

Polypeptides described herein can be isolated from naturally-occurring sources (e.g., isolated from a bacteriophage or a bacterial species, such as in particular a thermophilic bacteriophage or bacterium). Alternatively, the polypeptides can be chemically synthesized or recombinantly produced using the nucleic acids sequences of the present invention. For example, PCR primers can be designed to amplify an open reading frame (ORF) from the start codon to stop codon, e.g. using DNA of a suitable source organism or respective recombinant clones as a template. The primers can contain suitable restriction sites for efficient cloning into a suitable expression vector. The PCR product can be digested with the appropriate restriction enzyme and ligated between the corresponding restriction sites in the vector (the same restriction sites, or restriction sites producing the same cohesive ends or blunt end restriction sites).

A polypeptide of the present invention may be a viral polypeptide. For example, the viral source may be a bacteriophage having a bacterial such as E. coli or a thermophilic bacteriophage having a thermophilic bacterial host such as a Rhodothermus species, a Thermus species or Bacillus species. The viral source may also be a virus having a Eukaryotic host. The viral source may also be a prophage or other provirus with its genome integrated into that of the host.

Polypeptides described herein may be produced from any of a variety of microorganisms, either microorganisms that naturally contain in their genome nucleic acid sequences encoding the polypeptides of the invention or microorganisms into which a nucleic acid has been inserted, which encodes a polypeptide of the invention.

A polypeptide of the present invention may be a bacterial polypeptide. For example, the bacterial source may be a gram positive bacteria such as Bacillus, e.g. Bacillus stearothermophilus, Bacillus megaterium or Bacillus thuringiensis; or Streptomyces, e.g. Streptomyces lividans; or a gram negative bacterium such as E. coli, Pseudomonas sp.; Thermus, e.g. Thermus aquaticus, Thermus thermophilus or Thermus scotoductus or a Rhodothermus species; e.g. Rhodothermus marinus.

A polypeptide of the present invention may be obtained from an Archaea such as a Sulfolobus species, e.g. Sulfolobus acidocaldarius or Sulfolobus solfataricus; a Pyrobaculum species, e.g. Pyrobaculum islandicum or Pyrobaculum aerophilum; a Methanococcus species or a Halobacterium species.

A polypeptide of the present invention may be obtained from a microorganisms isolated from nature, e.g. from water or soil, including unclassified microorganisms or uncultivable or previously uncultured microorganisms, such as from environmental samples.

A polypeptide of the present invention may be encoded by a gene in an extrachromosomal genetic element such as a plasmid, including plasmids found in bacteria such as Thermus species.

A polypeptide of the present invention may be obtained from a non-bacterial source including eukaryotic organisms such as Fungi, including yeast; plants and animals.

A polypeptide of the present invention may obtained using nucleic add probes designed to identify and clone DNA encoding polypeptides having 5′-kinase and/or 3′-phosphatase activity using methods known in the art. A polypeptide of the invention can thus be obtained from a different genus or species, including from DNA isolated directly from environmental samples or DNA identified from screening genomic or cDNA libraries. In a preferred embodiment, a nucleic acid probe is a nucleic add sequence of the present invention shown as SEQ ID NO: 1 or a nucleic acid which encodes the polypeptide of the invention shown as SEQ ID NO: 2, or a subsequence thereof encoding an active fragment. A nucleic add probe may also be a degenerate probe designed from analysis of multiple sequences of polypeptides homologous to polypeptides of the present invention.

Polypeptides of the present invention can be used as a molecular weight marker on SDS-PAGE gels or on molecular sieve gel filtration columns using art-recognized methods. They are particularly useful for molecular weight markers for analysis of proteins from thermophilic organisms, as they will behave similarly (e.g., they will not denature as proteins from mesophilic organisms would).

The polypeptides of the present invention can be isolated or purified (e.g., to homogeneity) from cell culture (e.g., from culture of bacteria) by a variety of processes. These include, but are not limited to, anion or cation exchange chromatography, ethanol precipitation, affinity chromatography and high performance liquid chromatography (HPLC). The particular method used will depend upon the properties of the polypeptide; appropriate methods will be readily apparent to those skilled in the art. For example, with respect to protein or polypeptide identification, bands identified by gel analysis can be isolated and purified by HPLC, and the resulting purified protein can be sequenced. Alternatively, the purified protein can be enzymatically digested by methods known in the art to produce polypeptide fragments which can be sequenced. The sequencing can be performed, for example, by the methods of Wilm, et al. (Nature, 379:466-469 (1996)). The protein can be isolated by conventional means of protein biochemistry and purification to obtain a substantially pure product, i.e., 80, 95 or 99% free of cell component contaminants, as described in Jacoby, Methods in Enzymology, Volume 104, Academic Press, New York (1984); Scopes, Protein Purification, Principles and Practice, 2nd Edition, Springer-Verlag, New York (1987); and Deutscher (ed.), Guide to Protein Purification, Methods in Enzymology, Vol. 182 (1990).

The references cited herein are incorporated by reference in their entirety. While this invention has been particularly shown and described with references to preferred embodiments thereof, it will be understood by those skilled in the art that various changes in form and details may be made therein without departing from the spirit and scope of the invention as defined by the appended claims.

The following Examples are offered for the purpose of illustrating the present invention and are not to be construed to limit the scope of this invention.

### EXAMPLES

### Example 1

### Sequence Analysis, Over-Expression and Purification of RM378 PNK

The initial screening of the RM378 bacteriophage genome (accession number NC 004735) using standard BLAST analysis (Altschul, S. F. et al., (1990) J Mol Biol 215, 403-410) identified the RnIA gene encoding a homolog to the RNA ligase 1 in the T4 phage (Blondal, T. et al., (2003) Nucleic Acids Res 31, 7247-7254), but did not identify an ORF with similarity to the T4 PNK (pseT) gene. It was suspected that a gene encoding some form of a polynucleotide kinase was present in the RM278 genome, although the RNA degradation system in R. marinus could be very different from E. coli, given how distantly related these two bacteria are (Andresson, O. S., and Fridjonsson, O. H. (1994) J Bacteriol 176, 6165-6169). In subsequent and a more specific search, looking specifically for a T4 like 5′ kinase domain, we identified a putative PNK gene within the RM378 genome. The gene was 1086 bp in length and coded for a 361 amino acids polypeptide with a calculated mass 42,1 kDa with a theoretical pI of 7.3. The putative polynucleotide kinase gene designated pnkp (accession number NP_{—}835697) had in the initial analysis shown similarity, mainly to poly(A) polymerases from eubacteria, without clear indication of homology to T4 PNK. The identification was complicated by the fact that only the C-terminal half of this putative PNK gene product showed limited sequence similarity to the kinase domain located in the N-terminal half of T4 PNK. No significant similarity was seen with the C-terminal phosphohydrolase domain of T4 PNK. However sequence similarity searches with the N-terminal half of pnkp, showed similarity to the superfamily of HD metal dependent phosphohydrolase (Aravind, L., and Koonin, E. V. (1998) Trends Biochem Sci 23, 469-472), indicating a potential 3′ phosphohydrolase function, possibly analogous to the 3′ phosphatase function of T4 PNK.

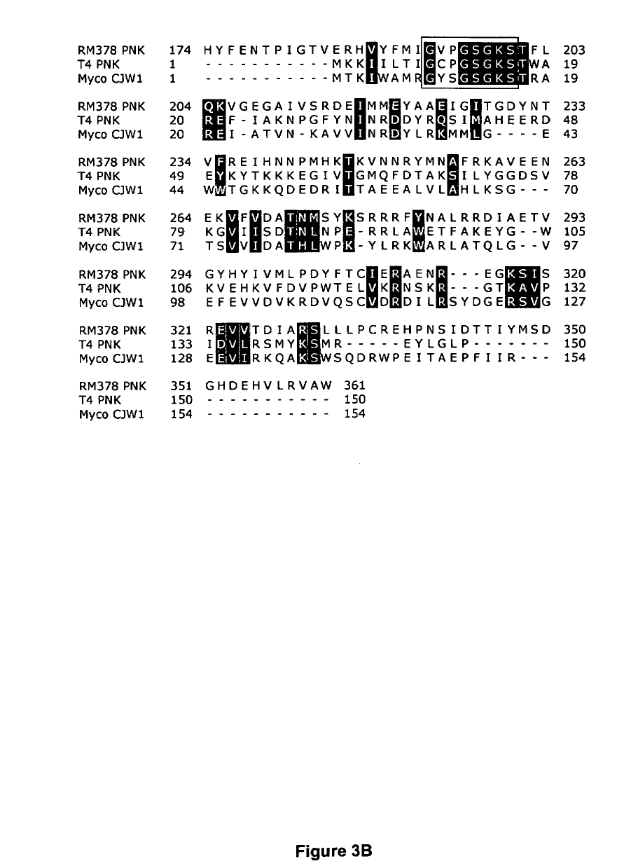

Amino acid sequence of the RM378 PNK 5′ kinase domain was compared to T4 and ACNV 5′ kinase domains as seen in FIG. 3A. Overall similarity is low but the characteristic P-loop motif is present. The RM378 PNK 3′ phosphohydrolase domain was compared to Clostridium acetobutylicum HD hydrolase and Desulfitobacterium hafniense tRNA nucleotidyltransferase/poly(A) polymerase as seen in FIG. 3B. As before the similarity is low but the HD box, which is the main characteristic for the superfamily is present (Aravind, L., and Koonin, E. V. (1998) Trends Biochem Sci 23, 469-472). It was our assumption that this HD phosphohydrolase domain might have similar activity as the T4 PNK family like phosphohydrolase even if they have no sequence similarity. The sequence analysis was followed by cloning and expression of the gene for biochemical characterization of the gene product.

All standard molecular biology protocols in this example and the following examples were done as described by Sambrook et al (Sambrook, J., Fritsch, E. F., and T., M. (1989) Molecular cloning, a laboratory manual, Cold Spring Harbor Laboratory, Cold Spring Harbor N.Y.). Chemicals, and media were purchased from Sigma Chemicals Inc. or Merck Inc., unless otherwise noted. Oligonucleotides were purchased from MWG Biotech Inc. and Eurogentech Inc. The putative pseT gene was amplified from the RM378 viral genome by standard PCR from RM378 viral DNA using primers KinR-ase-F: d(CCAATTGATTAATATGCCGAACTTCATTACAAACATC) and KinR-Bam-R: d(CGCGGATCCAAGCTACTCTCAACACAT) with Dynazyme™ DNA polymerase (Finnzymes Oy) as recommended by the manufacturer.

The PNK PCR product was cloned into pJOE vector with a connecting His tail to the C-terminal. Five clones were sequenced for verification of the DNA sequence and a vector-pnk clone, named pJOE-PNK, selected for expression experiments. The pJOE-PNK was transformed into CodonPlus® BL21 RIL E. coli cells (Stratagene Inc.).

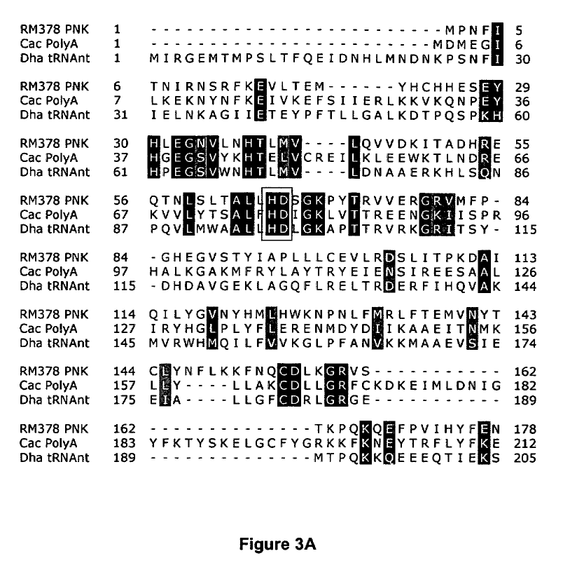

The His-tagged gene product was over-expressed in E. coli and purified to near homogeneity using nickel affinity chromatography. The strain was cultivated at 37° C. in a 10 L BioFlow 3000 fermentor and the expression induced with 1 mM IPTG. The cells were harvested and disrupted by sonication. The crude cell extract was centrifuged in a SA-600 rotor (Sorvall Inc.), at 10,400 g for 1 hour. The supernatant was collected and purified on XK 26/10 50 ml His Column (Amersham BioTech Inc.), packed with chelating sepharose, charged with nickel ions. The recombinant RM378 PNK protein was washed with washing buffer (10 mM sodium phosphate buffer pH 7.5, 0.5 M NaCl and 25 mM imidazole). Elution was preformed step wise (15%, 30% and 40%), in the same buffer with 500 mM imidazole. The eluted protein was then put through HiPrep sephacryl 26/60 S200 High Resolution column (Amersham Biotech Inc.) and eluted in 2× kinase storage buffer (20 mM Tris pH 9, 100 mM KCl, 2 mM DTT, 0.2 mM EDTA and 0.4 μM ATP), and 1:1 volume 100% glycerol added and put at −20° C. Aliquots from the purification procedure, were collected and run on 12% SDS-PAGE and stained with coomassie blue R-250 (Hames, B. D., and Rickwood, D. (1990) Gel Electrophoresis of Proteins. The Practical Approch Series, IRL Press, Oxford). The purification was estimated over 95% by SDS-PAGE analysis (FIG. 2). The recombinant PNK protein was run on gel chromatography to evaluate the oligomeric state of the protein. The results were inconclusive as numerous peaks were collected of the column ranging in size from 34 to 200 kDa and all displaying 5′ kinase activity (data not shown).

### Example 2

### Characterization of the PNK Recombinant Enzyme—5′ Kinase Activity

The standard PNK assay developed by Richardsson (Richardson, C. C. (1965) Proc Natl Acad Sci USA 54, 158-165) that measures conversion of γ-32P-ATP, was used for characterization of the RM378 PNK with some minor modifications. Standard reaction conditions were: 50 mM MOPS buffer pH 8.5, 1 mM DTT, 10 mM MgCl2, 25 μg/ml BSA, 1 mM spermidine and 5% PEG6000, 100 μM ATP (mixture of normal and γ-32P-ATP) and 0.5 mg/ml partial mircococcal nuclease digested calf thymus DNA or 50-100 μM DNA/RNA oligomers, and 0.0001-0.001 mg/ml PNK enzyme, incubated at 70° C. for 15-30 minutes.

The enzyme activity was determined in MOPS buffers at different pH values under standard conditions as described above. The apparent pH optimum was around 8.5 but good activity was observed between pH 8 and 9 (FIG. 3A).

Temperature optimum was determined by running the standard assay at different temperatures for one hour. The results showed that the apparent temperature optimum of the enzyme was about 70° C. under the given conditions (FIG. 3B). To determine the stability of the enzyme at different temperatures as a function of time, the enzyme was assayed at 50, 60, 65 and 70° C. and samples taken for analysis at time-points 0, 30, 60 and 120 min. The results showed linear increase in activity at 50, 60 and 65° C. but at 70° C. the activity of the enzyme started to decrease after an hour (FIG. 3C).

The effects of divalent cations were tested by running the standard assay in presence of Mg²⁺ or Mn²⁺ at different concentrations. The results showed that a divalent cation was essential for the 5′ kinase reaction and that maximum activity was reached in 10 and 100 μM for Mn²⁺ and Mg²⁺ respectively (data not shown). Activity was similar for Mn²⁺ and Mg²⁺ but Mn²⁺ concentration over 100 μM severely inhibited the reaction (data not shown). The effect of NaCl and KCl on the PNK activity was also studied and the results showing that both salts caused steady decrease in activity at concentrations higher than 10 mM (data not shown). Effect of spermidine was limited (data not shown) but PEG6000 greatly increased the activity of the enzyme (3-4 fold) at concentration between 5-10% but inhibited the 5′ kinase reaction at higher concentrations (FIG. 3D).

Effect of ADP was tested on three kind of reactions i) exchange reaction ii) 5′ kinase reaction and iii) dephosphosphorylation. This was done, by titrating different amounts of ADP to reaction mixtures containing i) 10 μM phosphorylated ssDNA oligo 10 μM γ-³²P-ATP, ii) 10 μM 5′ hydroxylated ssDNA oligomer with 10 μM γ-³²P-ATP, and iii) with 5′ ³²P-labeled ssDNA oligomer without ATP.

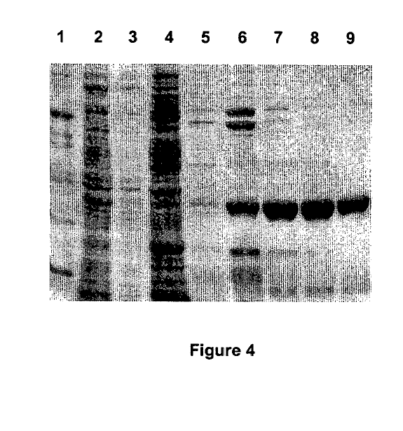

The effect of addition of ADP to 5′ kinase activity, dephosphorylation, and phosphate exchange was studied in controlled assay with 10:10 μM ATP and oligomer and by titrating the ADP concentration. As seen in FIG. 4A, the 5′ kinase activity decreased with increasing ADP concentration, with a complete inhibition at 50 μM ADP. If only ADP and ³²P-labeled oligomer were assayed with the PNK, the level of dephosphorylation increased as the ADP concentration was increased as seen in FIG. 4A. On the other hand, when labelling an already phosphorylated oligomer, the exchange reaction was overall about 5-8% independent of the ADP concentration, from 0 to 1 mM ADP.

Titration curves for ATP, r(A₂₀) and single stranded d(A₂₀) oligomers were done to calculate Kₘ values and find the maximum velocity of the 5′ kinase reaction. The results are shown for ATP in FIG. 4B and for, r(A₂₀) and d(A₂₀) in FIGS. 4C. The RM378 PNK had a Kₘ of 20 μM for ATP. The RM378 PNK does not discriminate between RNA and DNA oligomers in any degree, but the PNK showed somewhat better activity on ssDNA when compared to RNA. The Kₘ values were about 1.5 and 1.3 μM for r(A₂₀) and d(A₂₀), and the Vmax values were 160 and 220 μmolmg⁻¹h⁻¹ respectively. The 5′ kinase activity on blunt end double stranded DNA (micrococcal nuclease digested calf thymus DNA) was similar to that of single stranded DNA (data not shown).

To investigate the completeness of the 5′ kinase reaction, both r(A₂₀) and d(A₂₀) oligos at 20 μM concentration were labeled in a 10 μM ³²P labeled ATP mixture under optimal conditions. The results shown in FIG. 4D demonstrate that the ATP was depleted when labelling was done with limited amount of ATP. The RM378 PNK is therefore an excellent enzyme for nucleic acid labeling at elevated temperatures.

### Example 3

### Characterization of RM378 PNK Phosphohydrolase Activity

After determination of pH, cation and apparent temperature optimum, the standard phosphohydrolase assay was: Potassium acetate buffer pH 6.0, 5 mM MnCl₂, 1 mM DTT, 10 mM KCl₂, 0.1-5 mM CAMP and 0.05 mg/ml PNK. Reaction time was 30-60 minutes at 70-75° C. The reaction was quenched by adding 90 μL of Biomol Green reagent (Biomol Research Laboratories, Plymouth Meeting, Pa.) to 10 μL reaction. The release of phosphate was measured at A 620 nm in a Sunrise Absorbance Reader (Tecan Group Ltd, Maennedorf, Switzerland) and compare to a phosphate standard curve.

The characterization of the phosphohydrolase activity was done using two substrates: 3′-thymidine mono-phosphate (3′TMP) and cyclic 2′-3′cyclic adenosine mono-phosphate (cAMP). Determination of pH optimum was done with MOPS and potassium acetate buffers using 0.1 mM CAMP and 0.05 mg/ml PNK, apparent T optimum was also done under the standard condition by assaying at different temperatures. Protein concentration curve was done with CAMP at pH 6 in K-acetate buffer and different amount of PNK. Substrate concentration curves were done for CAMP and 3′TMP, under the standard conditions using different amount of substrates.

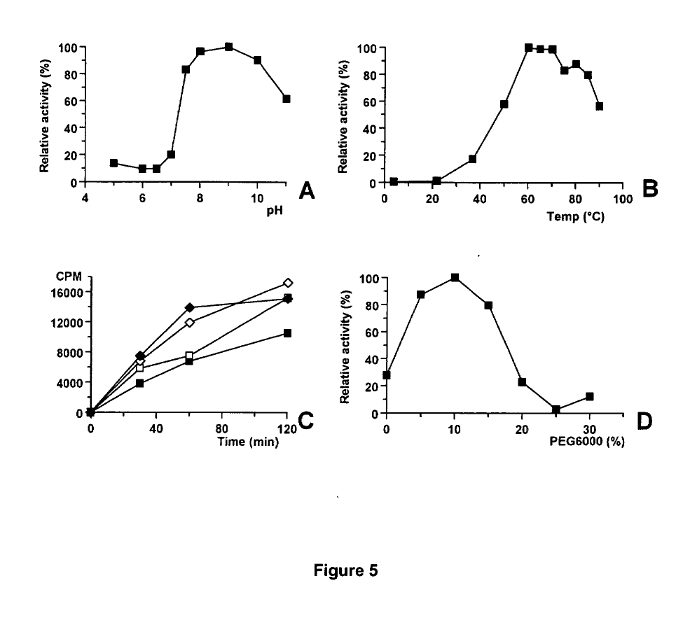

We suspected that cyclic phosphate might work better as a substrate for this potential HD phosphohydrolase, since the superfamily was found to be related the cNMP PDE family. It became apparent that the RM378 PNK had much more phosphohydrolase activity on CAMP compared to 3′TMP (FIG. 5D). We studied the pH profile and initially found using MOPS buffer that activity was only seen from pH 6-7, which is out of the MOPS stable pH range. We subsequently used potassium acetate buffer from pH 4-6 in the comparison and found out that the optimum was close to pH 6 with >40% activity from pH 5.5-7.0 as seen in FIG. 5A.

Mn²⁺ was superior to Mg²⁺ in the 3′ phosphohydrolase assay using CAMP as a substrate, with 5-10 fold higher activity at 1 mM concentration, as seen in FIG. 5D. Temperature optimum of the 3′ phosphohydrolase activity was determined as for the 5′ kinase activity, and resulted in apparent T optimum of 75° C. but the enzyme showed good activity (>50%) from 65-80° C. as seen in FIG. 5B. As before, the enzyme was stable for 2 hours at 65° C. for 2 hours showing linear accumulation of reaction product but started to loose activity at higher temperatures (data not shown). The CAMP and 3′TMP substrates were titrated in the 3′ phosphohydrolase reaction and Vₘₐₓ was 13.5 μmolmg⁻¹h⁻¹ and 1.5 μmolmg⁻¹h⁻¹ for CAMP and 3′TMP respectively. The Kₘ values for CAMP and 3TMP were 0.7 and 0.06 mM, respectively (FIG. 5C).

Comparison was done between RM378 and T4 PNK using CAMP, 3′TMP and d(A15)—PO₄⁻ oligomer as substrates, all in 0.1 mM concentration with 20 units of the enzymes in 20 μL reaction volumes using potassium acetate buffer pH 6 with both Mg²⁺ and Mn²⁺ as the divalent cation. The reactions were carried out for 2 hours at 37° C. and 70° C. for T4 and RM378 PNK, respectively. As seen in FIG. 5D, the two enzymes showed totally different substrate preference. While T4 PNK showed good activity on 3′-TMP and the oligomer 3′ phosphate group, the RM378 PNK revealed much better activity on the CAMP versus 3′-TMP and no detectable activity on the oligomer under the experimental conditions.

1. An isolated thermostable polypeptide having 5′-kinase activity and/or 3′-phosphatase activity.

2. The polypeptide of claim 1 having 5′-kinase and 3′-phosphatase activity.

3. The polypeptide of claim 1 obtainable from a bacteriophage.

4. The polypeptide of claim 1 selected from the group consisting of:
a) a polypeptide obtained from a bacteriophage capable of infecting thermophilic bacteria;
b) a polypeptide comprising the amino acid sequence of SEQ ID NO: 2;
c) a polypeptide encoded by the nucleic acid comprising the sequence of SEQ ID NO: 1;
d) a fragment or derivative of a), b) or c) which retains the 5′-kinase activity and/or 3′-phosphatase activity.

5. The polypeptide of claim 4 having 5′-kinase activity and comprising the amino acid sequence of residues 186-340 of SEQ ID NO: 2 or having substantial sequence identity thereto.

6. The polypeptide of claim 4 having 3′-phosphatase activity and comprising the amino acid sequence of residues 5-178 of SEQ ID NO: 2 or having substantial sequence identity thereto.

7. The polypeptide of claim 1 having an optimum 5′-kinase activity at a temperature in the range of 50-80° C.

8. The polypeptide of claim 2 having an optimum 3′-phosphatase activity at a temperature in the range of 50-80° C.

9. A fusion protein comprising the polypeptide of claim 1.

10-15. (canceled)

16. CA kit for transferring a phosphate group or phosphate group analogue from nucleotide triphosphate or a nucleotide analogue to the 5′ end of a nucleic acid or nucleic acid analog, said kit comprising:
a buffer medium or buffer components in a substantially dry form; and
a thermostable polypeptide of claim 1 having 5′-kinase activity.

17. (canceled)

18. The kit according to claim 16, further comprising one or more polypeptides having enzymatic activities selected from the group consisting of: DNA polymerase activity, RNA polymerase activity, DNA ligase activity, RNA ligase activity, phosphatase activity and exonuclease activity.

19. The kit according to claim 18 comprising a polypeptide having phosphatase activity for removal of 5′-phosphate group prior to phosphorylation using a thermostable polypeptide having 5′-kinase activity.

20. The kit according to claim 19 comprising a polypeptide having alkaline phosphatase activity.

21. The kit according to claim 19 comprising a heat-labile polypeptide having phosphatase activity.

22. The kit according to claim 16 further comprising a labeled nucleotide, for labeling nucleic acids with said labeled nucleotide.

23-33. (canceled)

34. A method of forming a phospho-monoester bond between a 5′-hydroxyl group of a nucleic acid and a phosphate in gamma position on a nucleotide or nucleotide analogue, comprising contacting a said 5′-hydroxyl group of a said nucleic acid and a said nucleotide triphosphate or nucleotide triphosphate analogue with an isolated thermostable polypeptide of claim 1 having 5′-kinase activity wherein a phospho-monoester bond is formed between the nucleic acid and the phosphate group.

35-47. (canceled)

48. A The method of claim 34 labeling nucleic acids, comprising: contacting a nucleic acid or a nucleic acid analog and a nucleotide triphosphate or nucleotide triphosphate analog, with a thermostable isolated polypeptide having 5′-kinase activity, wherein said polypeptide catalyzes the formation of a phosphomonoester bond between the nucleic acid or nucleic acid analog and the gamma phosphate of said nucleotide triphosphate or nucleotide triphosphate analog and wherein said gamma phosphate group is labeled for detection or labeled with a chemical group than can cause a secondary chemical reaction under suitable conditions.

49. The method of claim 48 wherein the labeling of the gamma phosphate is by means of a radioactive isotope.

50.-51. (canceled)

52. The method of claim 34 further comprising:
a) ligating said target nucleic acid into a cloning vehicle using a ligase;
b) transforming said cloning vehicle into a host organism of choice for production and plasmid preparation; and
c) optionally analyzing said target nucleic acid by restriction endonucleases, hybridization, blotting or DNA sequencing.

53.-67. (canceled)


SEQUENCE LISTING (8 sequences)

SEQ ID NO: 1 - DNA, 1083 bp, Bacteriohage RM378
atgccgaacttcattacaaacatcaggaattcccgttttaaggaagttctgaccgaaatgtaccattgccatcacgaaagcgagtaccaccttgagggaaatgttttaaatcacacgcttatggtattgcaggtggtagataagataaccgctgatcaccgggagcaaactaatctatccttaaccgcccttcttcatgatagtgggaaaccctatacccgtgttgtcgaaaggggaagagtaatgttccccggtcatgaaggggtgtctacgtatatcgctcctcttctgctgtgtgaagtattgagggattccctcatcacaccaaaagacgccattcaaatcctttacggcgtcaattaccatatgttgcactggaaaaatccaaacctttttatgcggcttttcaccgaaatggttaattatacctgtttatataacttcttgaaaaaattcaatcagtgtgatctaaagggtagggtttctacaaaaccccaaaagcaggaattccccgtaatccattattttgagaataccccgatcggtactgttgagcgccatgtttattttatgatcggggttccggggagtggaaagagcacgtttcttcagaaagttggagagggggcgattgtatcccgtgatgaaatcatgatggaatacgccgctgaaatagggatcacaggagactacaatactgttttccgggagattcacaacaaccctatgcataaaaccaaggtcaacaaccgctacatgaacgctttccgtaaggcggttgaagagaatgaaaaggtatttgtagacgcaaccaacatgagttataagagccggagacgtttttacaatgcgcttcggcgggatattgcggaaaccgtgggttaccattatatcgtaatgcttcccgattattttacgtgcattgaacgcgccgaaaatcgggaaggaaagtcgatttcaagggaagtggtaaccgatattgcgcggagtctgcttcttccgtgcagggaacatcccaacagcattgatacgacaatttatatgtctgatgggcatgatgaacatgtgttgagagtagcttgg

SEQ ID NO: 2 - protein, 361 aa, Bacteriophage RM378
  HDmotif (66)..(67): HD motif is characteristic of HD phophohydrolase family of enzymes
MPNFITNIRNSRFKEVLTEMYHCHHESEYHLEGNVLNHTLMVLQVVDKITADHREQTNLSLTALLHDSGKPYTRVVERGRVMFPGHEGVSTYIAPLLLCEVLRDSLITPKDAIQILYGVNYHMLHWKNPNLFMRLFTEMVNYTCLYNFLKKFNQCDLKGRVSTKPQKQEFPVIHYFENTPIGTVERHVYFMIGVPGSGKSTFLQKVGEGAIVSRDEIMMEYAAEIGITGDYNTVFREIHNNPMHKTKVNNRYMNAFRKAVEENEKVFVDATNMSYKSRRRFYNALRRDIAETVGYHYIVMLPDYFTCIERAENREGKSISREVVTDIARSLLLPCREHPNSIDTTIYMSDGHDEHVLRVAW

SEQ ID NO: 3 - protein, 363 aa, C. acetobutylicum
MDMEGILKEKNYNFKEIVKEFSIIERLKKVKQNPEYHGEGSVYKHTELVCREILKLEEWKTLNDREKVVLYTSALFHDIGKLVTTREENGKIISPRHALKGAKMFRYLAYTRYEIENSIREESAALIRYHGLPLYFLERENMDYDIIKAAEITNMKLLYLLAKCDLLGRFCKDKEIMLDNIGYFKTYSKELGCFYGRKKFKNEYTRFLYFKEKKIHPEAEMFDNRGFGVVAMMGLPLAGKDTYIKENFKNINVISLDDIREELNISSKRNSGKVAAIAISRAKQLLRRKESFIWNATNLRRENRQKLIRLCTAYGAKLKFIYLEVPYRELLSRNKMRSRYVPVEVINKMIRKMDMLEGEEICR

SEQ ID NO: 4 - protein, 205 aa, D. hafniense
MIRGEMTMPSLTFQEIDNHLMNDNKPSNFIIELNKAGIIETEYPFTLLGALKDTPQSPKHHPEGSVWNHTLMVLDNAAERKHLSQNPQVLMWAALLHDLGKAPTTRVRKGRITSYDHDAVGEKLAGQFLRELTRDERFIHQVAKMVRWHMQILFVVKGLPFANVKKMAAEVSIEEIALLGFCDRLGRGEMTPQKKQEEEQTIEKS

SEQ ID NO: 5 - protein, 301 aa, Enterobacteria phage T4
MKKIILTIGCPGSGKSTWAREFIAKNPGFYNINRDDYRQSIMAHEERDEYKYTKKKEGIVTGMQFDTAKSILYGGDSVKGVIISDTNLNPERRLAWETFAKEYGWKVEHKVFDVPWTELVKRNSKRGTKAVPIDVLRSMYKSMREYLGLPVYNGTPGKPKAVIFDVDGTLAKMNGRGPYDLEKCDTDVINPMVVELSKMYALMGYQIVVVSGRESGTKEDPTKYYRMTRKWVEDIAGVPLVMQCQREQGDTRKDDVVKEEIFWKHIAPHFDVKLAIDDRTQVVEMWRRIGVECWQVASGDF

SEQ ID NO: 6 - protein, 315 aa, Mycobacteriophage Cjw1
MTKIWAMRGYSGSGKSTRAREIATVNKAVVINRDYLRKMMLGEWWTGKKQDEDRITTAEEALVLAHLKSGTSVVIDATHLWPKYLRKWARLATQLGVEFEVVDVKRDVQSCVDRDILRSYDGERSVGEEVIRKQAKSWSQDRWPEITAEPFIIRPVGTISQLPPAIIVDIDGTLAHIPVNGRSPYDYTRVKEDKVDKEIAWLVSVLHQWRYVSDDDQAPEVIVMSGRDDTCRADTVEWMNQNDIPFDQLIMRPADAKDDRGNKLPDYQVKYQLFNDQIRDKYKVLFVLDDRQQVVDMWRKLGLKCLQVAPGELLT

SEQ ID NO: 7 - DNA, 37 bp, Artificial Sequence
  Sythentic primer KinR-ase-F
ccaattgattaatatgccgaacttcattacaaacatc

SEQ ID NO: 8 - DNA, 27 bp, Artificial Sequence
  Synthetic primer KinR-Bam-R
cgcggatccaagctactctcaacacat